(19) United States
(12) Patent Application Publication
(10) Pub. No.: US 2015/0250902 A1
(43) Pub. Date: Sep. 10, 2015

(54) pH-RESPONSIVE GADOLINIUM NANOPARTICLE CONJUGATES AND USES THEREOF

(71) Applicant: Colorado School of Mines, Golden, CO (US)

(72) Inventor: Stephen G. Boyes, Denver, CO (US)

(21) Appl. No.: 14/642,464

(22) Filed: Mar. 9, 2015

Related U.S. Application Data
(60) Provisional application No. 61/949,697, filed on Mar. 7, 2014.

(51) Int. Cl. A61K 49/00; A61K 49/04; A61K 49/18

(52) U.S. Cl. CPC A61K 49/0002; A61K 49/1881; A61K 49/0428; A61K 49/0438

(57) ABSTRACT

The present disclosure is directed generally to gadolinium nanoparticle conjugates, such as gold/gadolinium or iodine/gadolinium nanoparticle conjugates, nanoparticle conjugates including polymers, nanoparticle conjugates conjugated to targeting agents and pH responsive polymers, and their use in targeting, characterizing and/or imaging disease states in a patient.

### CROSS REFERENCE TO RELATED APPLICATIONS

This application claims priority to U.S. Provisional Patent Application No. 61/949,697, filed Mar. 7, 2014, and entitled “ph-Responsive Gadolinium Nanoparticle Conjugates and Uses Thereof,” which is hereby incorporated by reference in its entirety.

This application is related to U.S. patent application Ser. No. 13/202,311 filed Aug. 18, 2011, entitled “Gold/Lanthanide Nanoparticle Conjugates and Uses Thereof,” U.S. patent application Ser. No. 12/197,044 filed Aug. 22, 2008, entitled “Gold Nanoparticle Conjugates and Uses Thereof,” and U.S. patent application Ser. No. 12/197,061 filed Aug. 22, 2008, entitled “Lanthanide Nanoparticle Conjugates and Uses Thereof,” each of which is hereby incorporated by reference in its entirety.

### FIELD

The disclosure generally relates to multimodal imaging agents. Specifically, this disclosure relates to gadolinium nanoparticles with polymers grafted to, or polymerized from, the surface of a gadolinium metal organic framework. The nanoparticles also comprise an X-ray computed tomography contrast agent, such as iodine or gold. The polymers have functional groups that are derivativized to include imaging agents, pH responsive polymers, and targeting agents at their surface. Use of these multimodal nanoparticles will allow targeted delivery of imaging agents and pH responsive polymers to specific cells, tissues, and organs.

### BACKGROUND

Over the past few decades, medical imaging technologies have experienced dramatic growth and currently play a critical role in clinical oncology. (Rowe et al., (2009) Langmuir, 25:9487-9499.) However, the true potential of imaging in the clinical management of cancer patients has yet to be realized. With the advent of new and improved imaging techniques, such as molecular imaging, clinicians will not only be able to see where a tumor is located in the body, but also envisage biological activity of specific molecules that influence the response to therapy and behavior of various tumors. Access to such knowledge is predicted to have a major impact on cancer detection, diagnosis, therapeutic development, and personalized treatment, in addition to dramatically improving researchers' understanding of cancer.

One such area is the measurement of the extracellular pH (pHe) in solid tumors. For many years researchers have known the importance of the pHe in relation to cancer morbidity and mortality. It has been shown that a low pHe is associated with tumorigenic transformation, chromosomal rearrangements, induction of growth factors and proteases, extracellular matrix breakdown, and increased migration and invasion. (Hashim et al., NMR Biomed. (2011) 24:582-591.) A low pHe also has been identified as an important factor in producing more aggressive cancer phenotypes and causing metathesis of the primary carcinoma, both of which are leading causes of cancer morbidity and mortality. (Boyes et al., ACS Symp. Series (2010) 1053:65-101.) As such, the ability to measure the pHe of solid tumors using non-invasive and accurate techniques that also provide high spatiotemporal resolution has become increasingly important and is of great interest to clinicians.

There is currently no clinical method available for the in vivo determination of pHe in tumors and, despite the existing research in this area, in vivo applications have been limited due to problems with low changes in relaxivity limiting sensitivity, poor specificity due to lack of molecular targeting, solubility issues due to changes in ligand structure, and difficulty in tuning the pH response to match tumors. Thus, a non-invasive tool for accurately measuring pHe in vivo is needed.

### SUMMARY

The present disclosure is directed generally to nanoparticle conjugates, such as gadolinium (Gd) nanoparticle conjugates, nanoparticle conjugates including X-ray computed tomography (CT) contrast agents (CA), nanoparticle conjugates including polymers, conjugation to targeting agents and pH responsive polymers, and their use in targeting, imaging, and/or characterizing disease states in a patient. In certain embodiments, the gadolinium nanoparticle conjugates are multifunctional polymeric systems. Biocompatible polymer backbones that can be conjugated to imaging agents, targeting agents, and pH responsive polymers are produced. Post-polymerization modification of the polymer backbone allows attachment of targeting agents or pH responsive polymers to a functional group. The resulting gadolinium nanoparticle conjugates provide the ability to target, image, and characterize diseased cells.

In one aspect, the computed tomography contrast agent is gold (Au), and gold/gadolinium nanoparticle conjugates are provided. The conjugate includes a gadolinium metal organic framework (MOF) disposed on a gold nanoparticle with a polymer or polymer precursor containing a functional group grafted to the nanoparticle. A targeting agent and/or a pH responsive polymer is bonded to the polymer.

In another aspect, the computed tomography contrast agent is iodine (I) and iodine/gadolinium nanoparticle conjugates are provided. The conjugate includes an iodine nanoparticle coated with a gadolinium MOF and a polymer or polymer precursor containing a functional group grafted to the nanoparticle. A targeting agent and/or a pH responsive polymer is bonded to the polymer.

In another aspect, the disclosure is directed to a pharmaceutical composition comprising the gold/gadolinium or iodine/gadolinium nanoparticle conjugate as described herein, and a pharmaceutically acceptable carrier.

In a further aspect, the disclosure is directed to methods of making nanoparticle conjugates comprising disposing a gadolinium MOF on a nanoparticle comprising a computed tomography contrast agent and operably associating at least one pH responsive polymer with the nanoparticle. The computed tomography contrast agent can be iodine or gold.

In a further aspect, the disclosure is directed to methods of determining pHe in vivo comprising administering a gadolinium MOF nanoparticle comprising a computed tomography contrast agent and polymers bound to the gadolinium MOF nanoparticle to a patient. Some of the polymers can be pH responsive and some can be targeting agents. Longitudinal relaxation time is measured by MRI, and a change in relaxation time indicates pHe. The nanoparticles can also be quantified by computed tomography.

In further aspects, the present disclosure is directed to methods of determining pHe by administering a gold/gadolinium or iodine/gadolinium nanoparticle conjugate to a patient. The nanoparticle conjugate comprises pH responsive polymers. In various embodiments, a targeting agent localizes the nanoparticle conjugate to the site of the disease or disorder. The method can be further combined with imaging the nanoparticle conjugate at the disease location. Longitudinal relaxation time can be measured by MRI, and a change in relaxation time indicates pHe. The nanoparticles can also be quantified by computed tomography.

In still further aspects, the present disclosure is directed to methods of characterizing cells in a patient by administering a gold/gadolinium or iodine/gadolinium nanoparticle conjugate to a patient. Acidic pHe indicates diseased cells.

### BRIEF DESCRIPTION OF THE DRAWINGS

The disclosed Figures are exemplary, and are not intended to be limiting of the claims.

FIG. 1 depicts preparation of polymer modified surfaces by the (a) physisorption, (b) ‘grafting to’, and (c) ‘grafting from’ methods, (d) depicts a proposed coordination mechanism of attachment of thiolate polymer chain ends to metal organic frameworks (MOFs) of gadolinium (Gd) nanoparticles synthesized by a reverse microemulsion system employing the 1,4-BDC ligand.

FIG. 2 depicts the mushroom to brush spatial transition of polymers at a surface.

FIG. 3 depicts exemplary targeting molecules (folic acid or an RGD sequence) binding to a functionalized polymer grafted to a nanoparticle.

FIG. 4 depicts a ¹H NMR spectrum analysis of copolymer binding. (a) depicts a ¹H NMR spectrum of PNIPAM-co-PNAOS-co-PFMA copolymer, (b) depicts a ¹H NMR spectrum of folic acid, and (c) depicts a ¹H NMR spectrum of PNIPAM-co-PNAOS-co-PFMA copolymer reacted with folic acid.

FIG. 5 depicts fluorescence images of PNIPAM-co-PNAOS-co-PFMA modified gold/gadolinium (Au/Gd) nanoparticles.

FIG. 6 depicts transmission electron microscopy (TEM) images of (a) unmodified gold nanoparticles (Au NPs), (b) Au/Gd nanoparticles, and (c) PNIPAM homopolymer modified Au/Gd nanoparticles.

FIG. 7 depicts UV-Vis spectra of virgin gold nanoparticles, Au—Gd hybrid nanoparticles, and poly(N-isopropylacrylamide) modified Au—Gd hybrid nanoparticles.

FIG. 8 depicts ATR-FTIR (attenuated total reflection-fourier transform infrared) spectra of Au/Gd nanoparticles, poly(N-isopropylacrylamide) (PNIPAM) homopolymer synthesized via RAFT polymerization, and PNIPAM modified Au—Gd hybrid nanoparticles.

FIG. 9 depicts cell inhibition studies for unmodified gold nanoparticles and Au—Gd hybrid nanoparticles, homopolymer modified Au/Gd nanoparticles, and the reversible addition-fragmentation chain transfer (RAFT) copolymer, PNIPAM-co-PNAOS-co-PFMA.

FIG. 10 is a schematic diagram of a gadolinium nanoparticle surface-modified with pH-responsive polymers and targeting ligands according to one embodiment.

FIG. 11 depicts a mono-iodo ligand (left panel) and di-iodo ligand (right panel), and MRI and computed tomography images of iodine/gadolinium nanoparticles comprising the iodo ligands.

### DETAILED DESCRIPTION

The present disclosure relates to nanoparticles comprising gadolinium and an X-ray computed tomography (CT) contrast agent (CA), such as gold or iodine. In various aspects, the nanoparticles have applications as multimodal, targeting, and/or imaging agents for the determination of extracellular pH (pHe). The gadolinium can act as a positive computed tomography for magnetic resonance imaging (MRI), and the gold or iodine nanoparticle can be used as a computed tomography for computed tomography. Furthermore, these nanoparticles have been surface modified with pH-responsive polymers. Using the previously disclosed procedure for modifying gadolinium nanoparticles with reversible addition-fragmentation chain transfer (RAFT) polymerization polymers, cancer targeting ligands, such as folic acid and G-RGD sequences, and pH responsive polymers can be added to the surface of the nanoparticles to enable the targeted imaging and characterization of various cancers.

The gold or iodine nanoparticle is coated with a gadolinium metal organic framework (MOF) by disposing the gadolinium MOF on or onto the gold or iodine nanoparticle. The interaction between the gold or iodine nanoparticle and the MOF may include covalent as well as noncovalent interations. Without being limited by examples, the interactions between the nanoparticle and MOF may be ionic, hydrogen bonding, dipole-dipole, and Van der Waals forces.

### DEFINITIONS

A dash (“-”) that is not between two letters or symbols is used to indicate a point of attachment for a moiety or substituent. For example, —CONH₂ is attached through the carbon atom.

“Acyl” by itself or as part of another substituent refers to a radical —C(O)R₃₀, where R₃₀ is hydrogen, alkyl, cycloalkyl, cycloheteroalkyl, aryl, arylalkyl, heteroalkyl, heteroaryl, or heteroarylalkyl as defined herein. Representative examples include, but are not limited to, formyl, acetyl, cyclohexylcarbonyl, cyclohexylmethylcarbonyl, benzoyl, benzylcarbonyl, and the like.

“Acylamino” by itself or as part of another substituent refers to a radical —NR³¹C(O)R³², where R³¹ and R³² are independently hydrogen, alkyl, cycloalkyl, cycloheteroalkyl, aryl, arylalkyl, heteroalkyl, heteroaryl, or heteroarylalkyl as defined herein. Representative examples include, but are not limited to, formamido, acetamido, and benzamido.

“Acyloxy” by itself or as part of another substituent refers to a radical —OC(O)R³³, where R³³ is alkyl, cycloalkyl, cycloheteroalkyl, aryl, arylalkyl, heteroalkyl, heteroaryl, or heteroarylalkyl as defined herein. Representative examples include, but are not limited to, acetoxy, isobutyroyloxy, benzoyloxy, phenylacetoxy, and the like

“Alkanyl” by itself or as part of another substituent refers to a saturated branched, straight-chain, or cyclic alkyl radical derived by the removal of one hydrogen atom from a single carbon atom of a parent alkane. In some embodiments, an alkanyl group comprises from 1 to 20 carbon atoms. In other embodiments, an alkanyl group comprises from 1 to 10 carbon atoms. In still other embodiments, an alkanyl group comprises from 1 to 6 carbon atoms. Typical alkanyl groups include, but are not limited to, methanyl; ethanyl; propanyls such as propan-1-yl, propan-2-yl (isopropyl), cyclopropan-1l-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl” by itself or as part of another substituent refers to an unsaturated branched, straight-chain, or cyclic alkyl radical having at least one carbon-carbon double bond derived by the removal of one hydrogen atom from a single carbon atom of a parent alkene. The group may be in either the cis or trans conformation about the double bond(s). In some embodiments, an alkenyl group comprises from 1 to 20 carbon atoms. In other embodiments, an alkenyl group comprises from 1 to 10 carbon atoms. In still other embodiments, an alkenyl group comprises from 1 to 6 carbon atoms. Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkoxy” by itself or as part of another substituent refers to a radical—OR³⁴ where R³⁴ represents an alkyl or cycloalkyl group as defined herein. Representative examples include, but are not limited to, methoxy, ethoxy, propoxy, butoxy, cyclohexyloxy, and the like.

“Alkoxycarbonyl” by itself or as part of another substituent refers to a radical —C(O)—OR³⁵ where R³⁵ represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl, and the like.

“Alkoxycarbonylamino” by itself or as part of another substituent refers to a radical —NR³⁶C(O)—OR³⁷ where R³⁶ represents an alkyl or cycloalkyl group and R³⁷ is alkyl, cycloalkyl, cycloheteroalkyl, aryl, arylalkyl, heteroalkyl, heteroaryl, or heteroarylalkyl as defined herein. Representative examples include, but are not limited to, methoxycarbonylamino, tert-butoxycarbonylamino, and benzyloxycarbonylamino.

“Alkoxycarbonyloxy” by itself or as part of another substituent refers to a radical —OC(O)—OR³⁸ where R³⁸ represents an alkyl or cycloalkyl group as defined herein. Representative examples include, but are not limited to, methoxycarbonyloxy, ethoxycarbonyloxy, and cyclohexyloxycarbonyloxy.

“Alkyl” by itself or as part of another substituent refers to a saturated or unsaturated, branched, straight-chain, or cyclic monovalent hydrocarbon radical derived by the removal of one hydrogen atom from a single carbon atom of a parent alkane, alkene, or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

“Alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the expressions “alkanyl,” “alkenyl,” and “alkynyl” are used. In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms.

“Alkylamino” refers to a radical —NHR where R represents an alkyl or cycloalkyl group as defined herein. In certain embodiments, an alkoxy group is C₁₋₁₈ alkoxy, in certain embodiments, C₁₋₁₂ alkoxy, in certain embodiments, C₁₋₈ alkoxy, in certain embodiments, C₁₋₆ alkoxy, in certain embodiments, C₁₋₄ alkoxy, and in certain embodiments, C₁₋₃ alkoxy. Representative examples include, but are not limited to, methylamino, ethylamino, 1-methylethylamino, cyclohexyl amino, and the like.

“Alkylsulfinyl” refers to a radical —S(O)R where R is an alkyl or cycloalkyl group as defined herein. Representative examples include, but are not limited to, methylsulfinyl, ethylsulfinyl, propylsulfinyl, butylsulfinyl, and the like.

“Alkylsulfonyl” refers to a radical —S(O)₂R where R is an alkyl or cycloalkyl group as defined herein. Representative examples include, but are not limited to, methylsulfonyl, ethylsulfonyl, propylsulfonyl, butylsulfonyl, and the like.

“Alkylthio” refers to a radical —SR where R is an alkyl or cycloalkyl group as defined herein that may be optionally substituted as defined herein. Representative examples include, but are not limited to, methylthio, ethylthio, propylthio, butylthio, and the like.

“Alkynyl” by itself or as part of another substituent refers to an unsaturated branched, straight-chain, or cyclic alkyl radical having at least one carbon-carbon triple bond derived by the removal of one hydrogen atom from a single carbon atom of a parent alkyne. In some embodiments, an alkynyl group comprises from 1 to 20 carbon atoms. In other embodiments, an alkynyl group comprises from 1 to 10 carbon atoms. In still other embodiments, an alkynyl group comprises from 1 to 6 carbon atoms. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Amide derivatives” as used herein refer to compounds having the structure RC(O)NR′R″. The R, R′, and R″ in amide derivatives can each independently be any desired substituent, including but not limited to, hydrogen, halides, and substituted or unsubstituted alkyl, alkoxy, aryl, or acyl groups. The amine or amide group can be used as the point of attachment to a therapeutic or targeting group, optionally via a linker. Examples of amines and amides include, but are not limited to, 2-(N,N-diethylamino)ethyl methacrylate, 2-(N,N-diethylamino)ethyl acrylate, N42-(N,N-dimethylamino)ethyl]methacrylamide, N-[3-(N,N-dimethylamino)propyl]acrylamide, diallylamine, methacryloyl-L-lysine, 2-(tert-butylamino)ethyl methacrylate, N-(3-aminopropyl)methacrylamide hydrochloride, 3-dimethylaminoneopentyl acrylate, N-(2-hydroxypropyl)methacrylamide, N-methacryloyl tyrosine amide, 2-diisopropylaminoethyl methacrylate, 3-dimethylaminoneopentyl acrylate, 2-aminoethyl methacrylate hydrochloride, hydroxymethyldiacetoneacrylamide, N-(iso-butoxymethyl)methacrylamide, and N-methylolacrylamide.

“Amine derivatives” are compounds or radicals thereof having a functional group containing at least one nitrogen, and having the structure RNR′R″. R, R′ and R″ in amine derivatives can each independently be any desired substituent, including but not limited to, hydrogen, halides, and substituted or unsubstituted alkyl, alkoxy, aryl, or acyl groups.

“Anhydride derivatives” as used herein refer to compounds or radicals having the chemical structure R¹C(O)OC(O)R². The carboxyl groups, after optional removal of R¹ or R² groups, can be used as the point of attachment to a therapeutic or targeting group, optionally via a linker. Examples of anhydride derivatives include, but are not limited to, acrylic anhydride, methacrylic anhydride, maleic anhydride, and 4-methacryloxyethyl trimellitic anhydride

“Antibody” refers to a monomeric or multimeric protein comprising one or more polypeptide chains that binds specifically to an antigen. An antibody can be a full length antibody or an antibody fragment.

“Antibody, full length antibody,” herein refers to the structure that constitutes the natural biological form of an antibody, including variable and constant regions. For example, in most mammals, including humans and mice, the full length antibody of the IgG class is a tetramer and consists of two identical pairs of two immunoglobulin chains, each pair having one light and one heavy chain, each light chain comprising immunoglobulin domains V_{L} and C_{L}, and each heavy chain comprising immunoglobulin domains V_{H}, C_{H1} (Cγ1), C_{H2} (Cγ2), and C_{H3} (Cγ3). In some mammals, for example in camels and llamas, IgG antibodies may consist of only two heavy chains, each heavy chain comprising a variable domain attached to the Fc region.

“Antibody fragments” are portions of full length antibodies that bind antigens. Specific antibody fragments include, but are not limited to, (i) the Fab fragment consisting of V_{L}, V_{H}, C_{L}, and C_{H1} domains, (ii) the Fd fragment consisting of the V_{H} and C_{H1} domains, (iii) the Fv fragment consisting of the V_{L} and V_{H} domains of a single antibody; (iv) the dAb fragment (Ward et al., 1989, Nature 341:544-546) which consists of a single variable, (v) isolated CDR regions, (vi) F(ab′)2 fragments, a bivalent fragment comprising two linked Fab fragments (vii) single chain Fv molecules (scFv), wherein a V_{H} domain and a V_{L} domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883), (viii) bispecific single chain Fv dimers (PCT/US92/09965) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448). In certain embodiments, antibodies are produced by recombinant DNA techniques. Other examples of antibody formats and architectures are described in Holliger & Hudson, 2006, Nature Biotechnology 23(9):1126-1136, and Carter 2006, Nature Reviews Immunology 6:343-357 and references cited therein, all expressly incorporated by reference. In additional embodiments, antibodies are produced by enzymatic or chemical cleavage of naturally occurring antibodies.

“Aromatic Ring System” by itself or as part of another substituent refers to an unsaturated cyclic or polycyclic ring system radical having a conjugated π electron system. Specifically included within the definition of “aromatic ring system” are fused ring systems in which one or more of the rings are aromatic and one or more of the rings are saturated or unsaturated, such as, for example, fluorene, indane, indene, phenalene, etc. Typical aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Aryl” by itself or as part of another substituent refers to a monovalent aromatic hydrocarbon radical derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In some embodiments, an aryl group is from 6 to 20 carbon atoms. In other embodiments, an aryl group is from 6 to 12 carbon atoms.

“Arylalkyl” by itself or as part of another substituent refers to an acyclic alkyl radical in which one of the hydrogen atoms bonded to a carbon atom, typically a terminal or sp3 carbon atom, is replaced with an aryl group. Typical arylalkyl groups include, but are not limited to, benzyl, 2-phenylethan-l-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the nomenclature arylalkanyl, arylalkenyl, and/or arylalkynyl is used. In some embodiments, an arylalkyl group is (C₆-C₃₀) arylalkyl, e.g., the alkanyl, alkenyl, or alkynyl moiety of the arylalkyl group is (C₁-C₁₀) and the aryl moiety is (C6-C20). In other embodiments, an arylalkyl group is (C₆-C₂₀) arylalkyl, e.g., the alkanyl, alkenyl, or alkynyl moiety of the arylalkyl group is (C₁-C₈) and the aryl moiety is (C₆-C₁₂).

“Aryloxy” refers to a radical —C—O-aryl where aryl is as defined herein.

“Aryloxycarbonyl” refers to a radical —C(O)—O-aryl where aryl is as defined herein.

“Azide derivatives” as used herein refer to compounds or radicals thereof having the structure N═N═N. The azide group can be used as the point of attachment to a therapeutic or targeting group, optionally via a linker. Examples of azide derivatives include, but are not limited to, 2-hydroxy-3-azidopropyl methacrylate, 2-hydroxy-3-azidopropyl acrylate, and 3-azidopropyl methacrylate.

“Carbamoyl” by itself or as part of another substituent refers to the radical —C(O)NR³⁹R⁴⁰ where R³⁹ and R⁴⁰ are independently hydrogen, alkyl, cycloalkyl, or aryl as defined herein.

“Carbamoyloxy” by itself or as part of another substituent refers to the radical —OC(—)NR⁴¹R⁴² where R⁴¹ and R⁴² are independently hydrogen, alkyl, cycloalkyl, or aryl as defined herein.

“Carbazole derivatives” as used herein refer to compounds or radicals thereof having the structure

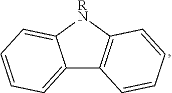

and any substitutions at any site thereof. The carbazole group can be used as the point of attachment to a therapeutic or targeting group, optionally via a linker. Examples of carbazole derivatives include but are not limited to, N-vinylcarbazole.

“Carboxylate” refers to a compound or radical thereof having the structure RCOO—, where R can be any desired substitutent.

“Compounds” include, but are not limited to, optical isomers of compounds, racemates thereof, and other mixtures thereof. In such embodiments, the single enantiomers or diastereomers, i.e., optically active forms, can be obtained by asymmetric synthesis or by resolution of the racemates. Resolution of the racemates may be accomplished, for example, by conventional methods such as crystallization in the presence of a resolving agent, or chromatography, using, for example a chiral high-pressure liquid chromatography (HPLC) column In addition, compounds can include Z- and E-forms (or cis- and trans-forms) of compounds with double bonds.

“Compounds” may also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms and as N-oxides. In general, compounds as referred to herein may be free acid, hydrated, solvated, or N-oxides of a Formula. Certain compounds may exist in multiple crystalline, co-crystalline, or amorphous forms. Compounds include pharmaceutically acceptable salts thereof, or pharmaceutically acceptable solvates of the free acid form of any of the foregoing, as well as crystalline forms of any of the foregoing.

“Compounds” as defined by a chemical formula as disclosed herein include any specific compounds within the formula. Compounds may be identified either by their chemical structure and/or chemical name. When the chemical structure and chemical name conflict, the chemical structure is determinative of the identity of the compound. The compounds described herein may comprise one or more chiral centers, and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan.

“Conjugate acid of an organic base” refers to the protonated form of a primary, secondary, or tertiary amine or heteroaromatic nitrogen base. Representative examples include, but are not limited to, triethylammonium, morpholinium, and pyridinium.

“Covalent grafting” as used herein refers to attaching a polymer, polymer precursor, or small molecule by one or more covalent bonds from a functional group to the surface of a nanoparticle or by a delocalized bond complex, such as a delocalized bond complex.

“Cyano derivatives” as used herein refer to compounds or radicals thereof having the structure RCN. R can each independently be any desired substituent. The cyano group can be used as the point of attachment to a therapeutic or targeting group, optionally via a linker. Examples of cyano derivatives include, but are not limited to, 2-cyanoethyl acrylate.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. In some embodiments, the cycloalkyl group is (C₃-C₁₀) cycloalkyl. In other embodiments, the cycloalkyl group is (C₃-C₇) cycloalkyl.

“Cycloheteroalkyl” by itself or as part of another substituent refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine, and the like.

“Dialkylamino” by itself or as part of another substituent refers to the radical —NR⁴³R⁴⁴ where R⁴³ and R⁴⁴ are independently alkyl, cycloalkyl, cycloheteroalkyl, arylalkyl, heteroalkyl, or heteroarylalkyl, or optionally R⁴³ and R⁴⁴ together with the nitrogen to which they are attached form a cycloheteroalkyl ring.

“Epoxide derivatives” as used herein refer to compounds or radicals thereof having the following chemical structure:

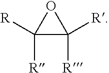

The epoxide group can be used as the point of attachment to a therapeutic or targeting group, optionally via a linker. Examples of epoxide derivatives include, but are not limited to, glycidyl methacrylate.

“Ester derivatives” as used herein refer to compounds or radicals thereof having the generic chemical structure RC(O)OR′. R and R′ can each independently be any desired substituent. The ester group can be used as the point of attachment to a therapeutic or targeting group, optionally via a linker. Examples include, but are not limited to, methyl acrylate, methyl methacrylate, tert-butyl acrylate, tert-butyl methacrylate, vinyl acetate, benzyl acrylate, and benzyl methacrylate.

“Ether derivatives” as used herein refer to compounds or radicals thereof having the generic chemical structure R—O—R′. The ether group can be used as the point of attachment to a therapeutic or targeting group, optionally via a linker. Examples include, but are not limited to, methyl vinyl ether, butyl vinyl ether, 2-chloroethyl vinyl ether, and cyclohexyl vinyl ether.

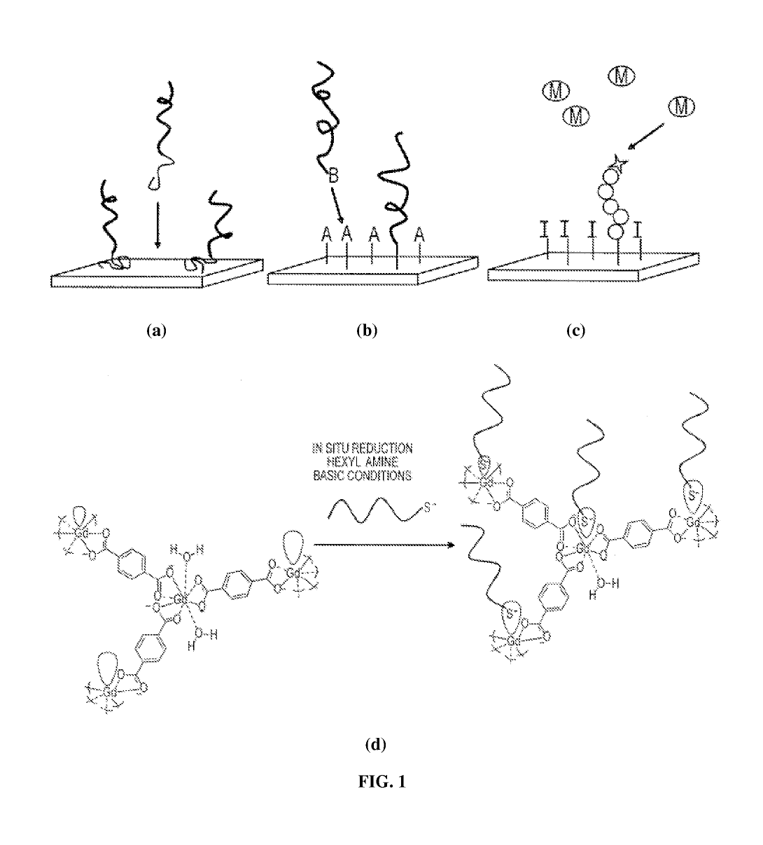

“Grafting” or “grafted onto” as used herein refers to attaching a polymer, polymer precursor, or small molecule to the surface of a nanoparticle via a single functional group. Grafting includes both covalent and non-covalent binding, as well as, but not limited to, delocalized bond formation between one or more atoms of the nanoparticle and one or more atoms of the functional group, ionic bonding, hydrogen bonding, dipole-dipole bonding, and van der Waals forces (for non-limiting examples see FIG. 1). Formation of exemplary bonds are depicted in Schemes 2 and 3 described herein. The terms “grafting” and “grafting onto” include methods conventionally referred to as grafting from and grafting to.

“Halide derivatives” as used herein refer to compounds or radicals thereof having a halide substituent. The halide group can be used as the point of attachment to a therapeutic or targeting group, optionally via a linker. Examples include, but are not limited to, vinyl chloride, 3-chlorostyrene, 2,4,6-tribromophenyl acrylate, 4-chlorophenyl acrylate, and 2-bromoethyl acrylate. Non-limiting examples include, but are not limited to, divinylbenzene, ethylene glycol diacrylate, N,N-diallylacrylamide, and allyl methacrylate.

“Halo” means fluoro, chloro, bromo, or iodo radical.

“Heteroalkyl, Heteroalkanyl, Heteroalkenyl, and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, S—, —O—O—, —S—S—, —O—S—, —NR⁴⁵ R46, —═N—N═—, —N═N—, —N═N—NR³⁷R⁴⁸, —PR⁴⁹—, —P(O)₂—, —POR⁵⁰—, —O—P(O)₂—, —SnR⁵¹R⁵²—, and the like, where R⁴⁵, R⁴⁶, R⁴⁷, R⁴⁸, R⁴⁹, R⁵⁰, R⁵¹, and R⁵² are independently hydrogen, alkyl, subsituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, or substituted heteroarylalkyl.

“Heteroalkyloxy” means an —O-heteroalkyl where heteroalkyl is as defined herein.

“Heteroaromatic Ring System” by itself or as part of another substituent refers to an aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl” by itself or as part of another substituent refers to a monovalent heteroaromatic radical derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. The heteroaryl group may be a 5-20 membered heteroaryl, or may be a 5-10 membered heteroaryl. Certain heteroaryl groups are those derived from thiophene, pyrrole, benzothiophene, benzofuran, indole, pyridine, quinoline, imidazole, oxazole, and pyrazine.

“Heteroarylalkyl” by itself or as part of another substituent refers to an acyclic alkyl radical in which one of the hydrogen atoms bonded to a carbon atom, typically a terminal or sp3 carbon atom, is replaced with a heteroaryl group. Where specific alkyl moieties are intended, the nomenclature heteroarylalkanyl, heteroarylalkenyl, and/or heterorylalkynyl is used. In some embodiments, the heteroarylalkyl group is a 6-30 membered heteroarylalkyl, e.g., the alkanyl, alkenyl, or alkynyl moiety of the heteroarylalkyl is 1-10 membered and the heteroaryl moiety is a 5-20-membered heteroaryl. In other embodiments, the heteroarylalkyl group is a 6-20 membered heteroarylalkyl, e.g., the alkanyl, alkenyl, or alkynyl moiety of the heteroarylalkyl is 1-8 membered and the heteroaryl moiety is a 5-12-membered heteroaryl.

“Heteroaryloxycarbonyl” refers to a radical —C(O)—OR where R is heteroaryl as defined herein.

“Hydroxyl derivative” as used herein refers to a compound or radical having the structure ROH. The deprotonated hydroxyl group can be used as the point of attachment to a therapeutic or targeting group, optionally via a linker. Example of hydroxyl derivatives include, but are not limited to, vinyl alcohol, 2-hydroxyethyl acrylate, 2-hydroxyethyl methacrylate, 2-allyl-2-methoxyphenol, divinyl glycol, glycerol monomethacrylate, poly(propylene glycol) monomethacrylate, N-(2-hydroxypropyl)methacrylamide, hydroxymethyldiacetoneacrylamide, poly(ethylene glycol) monomethacrylate, N-methacryloylglycylglycine, N-methacryloylglycyl-DL-phenylalanylleucylglycine, 4-methacryloxy-2-hydroxybenzophenone, 1,1,1-trimethylolpropane diallyl ether, 4-allyl-2-methoxyphenol, hydroxymethyldiacetoneacrylamide, N-methylolacrylamide, and sugar based monomers.

“Maleimide derivative” as referred to herein refers to a compound or a radical thereof having the structure:

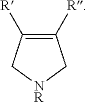

“Morpholine derivatives” as used herein refer to compounds or radicals thereof having the structure:

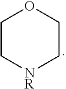

Typically, the amine group serves as the point of attachment to other compounds. The morpholine group can be used as the point of attachment to a therapeutic or targeting group, optionally via a linker. Examples of morpholine derivatives include, but are not limited to, N-acryloylmorpholine, 2-N-morpholinoethyl acrylate, and 2-N-morpholinoethyl methacrylate.

“Nanoparticle” as referred to herein means a particle having at least one special dimension measurable less than a micron in length. Nanoparticles include conventionally known nanoparticles such as nanorods, nanospheres, and nanoplatelets. In various embodiments, for example, nanospheres can be a rod, sphere, or any other three dimensional shape. Nanoparticles are generally described, for example, in Burda et al., Chem. Rev. 2005, 105, 1025-1102.

“Nitro derivatives” as used herein refer to compounds or radicals thereof having an NO₂ group. The nitro group can be used as the point of attachment to a therapeutic or targeting group, optionally via a linker. Examples include, but are not limited to, o-nitrobenzyl methacrylate, methacryloylglycyl-DL-phenylalanyl-L-leucyl-glycine 4-nitrophenyl ester, methacryloylglycyl-L-phenylalanyl-L-leucyl-glycine 4-nitrophenyl ester, N-methacryloylglycylglycine 4-nitrophenyl ester, and 4-nitrostyrene.

“Patient” includes animals and mammals, such as for example, humans.

“Pharmaceutical composition” refers to a compound or nanoparticle and at least one pharmaceutically acceptable vehicle, with which the compound or nanoparticle is administered to a patient.

“Pharmaceutically acceptable salt” refers to a salt of a compound, which possesses the desired pharmacological activity of the parent compound. Such salts include acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like. In certain embodiments, a pharmaceutically acceptable salt is the hydrochloride salt. In certain embodiments, a pharmaceutically acceptable salt is the sodium salt.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

“Phosphate derivatives” as used herein refer to compounds or radicals thereof having at least one compound containing the structure RR′R″PO₄. R, R′ and R″ can each independently be any desired substituent, including but not limited to hydrogen, alkyl, alkoxy, aryl, or acyl groups. The phosphate group can be used as the point of attachment to a therapeutic or targeting group, optionally via a linker. Examples of phosphate derivatives include, but are not limited to, monoacryloxyethyl phosphate and bis(2-methacryloxyethyl)phosphate.

“Phosphinate” refers to a compound or radical thereof having the structure OP(OR)R′R″ where R, R′, and R′ can each independently be any desired substituent.

“Phosphonate” refers to a compound or radical thereof having the structure R—PO(OH)₂ or R—PO(OR′)₂ where R and R′ can each independently be any desired substituent.

“R, R′, R″, and R′″ can each independently be any desired substituent.

“Reducing agent” is an element or a compound that reduces another species. Exemplary reducing agents include, but are not limited to, ferrous ion, lithium aluminium hydride (LiAlH₄), potassium ferricyanide (K₃Fe(CN)₆), sodium borohydride (NaBH₄), sulfites, hydrazine, diisobutylaluminum hydride (DIBAH), primary amines, and oxalic acid (C₂H₂O₄).

“Salt” refers to a salt of a compound, including, but not limited to, pharmaceutically acceptable salts.

“Silane derivative” as used herein refers to compounds or radicals thereof having at least one substituent having the structure RSiR′R″R′″. R, R′, and R″ can each independently be any desired substituent, including but not limited to hydrogen, alkyl, alkoxy, aryl, or acyl groups. The silane group can be used as the point of attachment to a therapeutic or targeting group, optionally via a linker. Examples of silane derivatives include, but are not limited to, 3-methacryloxypropyl trimethoxysilane, vinyltriethoxysilane, 2-(trimethylsiloxy)ethyl methacrylate, and 1-(2-trimethylsiloxyethoxy)-1-trimethylsiloxy-2-methylpropene.

“Solvate” refers to a molecular complex of a compound with one or more solvent molecules in a stoichiometric or non-stoichiometric amount. Such solvent molecules are those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water.

“Substituted” refers to a group in which one or more hydrogen atoms are each independently replaced with the same or different substituent(s). Typical substituents include, but are not limited to, —X, —R²⁹, —O—, ═O, —OR²⁹, —SR²⁹, —S—, ═S, —NR²⁹R³⁰, ═NR²⁹, —CX₃, —CF₃, —CN, —OCN, —SCN, —NO, —NO₂, ═N₂, —N₃, —S(O)₂OH, —S(O)₂OH, —S(O)₂R²⁹, —OS(O₂)O—, —OS(O)₂R²⁹, —P(O)(O—)₂, —P(O)(OR²⁹)(O—), —OP(O)(OR²⁹)(OR³⁰), —C(O)R²⁹, —C(S)R²⁹, —C(O)OR²⁹, —C(O)NR²⁹R³⁰, —C(O)O—, —C(S)OR²⁹, —NR³¹C(O)NR²⁹R³⁰, —NR³¹C(S)NR²⁹R³⁰, —NR³¹C(NR²⁹)NR²⁹R³⁰, and —C(NR²⁹)NR²⁹R³⁰, where each X is independently a halogen; each R²⁹ and R³⁰ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR³¹R³², —C(O)R³¹, or —S(O)₂R³¹, or optionally R²⁹ and R³⁰ together with the atom to which they are both attached form a cycloheteroalkyl or substituted cycloheteroalkyl ring; and R³¹ and R³² are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, or substituted heteroarylalkyl.

“Succinimide derivatives” as used herein refer to compounds or radicals thereof having the group

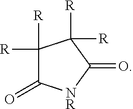

The succinyl R groups can be substituted by any substituent, for example,substituted or unsubstituted alkyl, alcoxy, and/or aryl groups. Typically, the succinimide group is attached to a compound via a covalent bond at the nitrogen. The succinimide group can be used as the point of attachment to a therapeutic or targeting group, optionally via a linker. A succinimide derivative can be a sulfo-containing succinimide derivative. N-acryloxysuccinimide is an exemplary succinimide derivative.

“Sulfonamido” by itself or as part of another substituent refers to a radical —NR⁵³S(O)₂R⁵⁴, where R⁵³ is alkyl, substituted alkyl, cycloalkyl, cycloheteroalkyl, aryl, substituted aryl, arylalkyl, heteroalkyl, heteroaryl, or heteroarylalkyl and R⁵⁴ is hydrogen, alkyl, cycloalkyl, cycloheteroalkyl, aryl, arylalkyl, heteroalkyl, heteroaryl, or heteroarylalkyl as defined herein. Representative examples include, but are not limited to, methanesulfonamido, benzenesulfonamido, and p-toluenesulfonamido.

“Sulfonic acids derivatives” as used herein are a class of organic acid radicals with the general formula RSO₃H or RSO₃. An oxygen, suflur, or R moiety can serve as a point of attachment. Sulfonic acid salt derivatives substitute a cationic salt (e.g. Na⁺, K⁺, etc.) for the hydrogen on the sulfate group. In various embodiments, the deprotonated sulfonic acid group can be used as the point of attachment to a therapeutic or targeting group, optionally via a linker. Examples of sulfonic acid derivatives and include, but are not limited to, 2-methyl-2-propane-1-sulfonic acid—sodium salt, 2-sulfoethyl methacrylate, 3-phenyl-1-propene-2-sulfonic acid—p-toluidine salt, 3-sulfopropyl acrylate—potassium salt, 3-sulfopropyl methacrylate—potassium salt, ammonium 2-sulfatoethyl methacrylate, styrene sulfonic acid, and 4-sodium styrene sulfonate.

“Sulphate” refers to a compound or radical thereof having the structure RSO₄ where R can be any desired substituent.

“Sulphonate” refers to a compound or radical thereof having the structure RSO₂O— where R can be any desired substituent.

“Therapeutically effective amount” or “effective amount” refers to the amount of a compound that, when administered to a subject for treating a disease or disorder, or at least one of the clinical symptoms of a disease or disorder, is sufficient to affect such treatment of the disease, disorder, or symptom. The “therapeutically effective amount” can vary depending, for example, on the compound, the disease, disorder, and/or symptoms of the disease or disorder, severity of the disease, disorder, and/or symptoms of the disease or disorder, the age, weight, and/or health of the patient to be treated, and the judgment of the prescribing physician. An appropriate therapeutically effective amount in any given instance may be ascertained by those skilled in the art or capable of determination by routine experimentation.

“Therapeutically effective dose” or “effective dose” refers to a dose of a drug, prodrug, or active metabolite of a prodrug that provides effective treatment of a disease or disorder in a patient. A therapeutically effective dose may vary from compound to compound and from patient to patient, and may depend upon factors such as the condition of the patient and the route of delivery. A therapeutically effective dose may be determined in accordance with routine pharmacological procedures known to those skilled in the art.

“Thioester” refers to a compound or radical thereof having the structure R—S—CO—R′, where R and R′ can each independently be any desired substituent.

“Thioether” refers to a compound or radical thereof having the structure R—S—CO—R, where R and R′ can each independently be any desired substituent.

“Thiolate” refers to a compound or radical thereof having the structure —SR, where R can be any desired substituent.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder, or at least one of the clinical symptoms of the disease or disorder, or reducing the risk of developing a disease, disorder, or at least one of the clinical symptoms of a disease or disorder. “Treating” or “treatment” also refers to inhibiting the disease, disorder, or at least one of the clinical symptoms of a disease or disorder, either physically, (e.g., stabilization of a discernible symptom), physiologically, (e.g., stabilization of a physical parameter), or both, and to inhibiting at least one physical parameter which may or may not be discernible to the patient. In certain embodiments, “treating” or “treatment” refers to delaying the onset of the disease or disorder or at least one or more symptoms thereof in a patient which may be exposed to or predisposed to a disease or disorder even though that patient does not yet experience or display symptoms of the disease or disorder.

### X-ray Computed Tomography Contrast Agent Nanoparticles

The presently disclosed nanoparticles comprise a computed tomography contrast agent, such as gold, iodine, barium, or a lanthanide. In some embodiments, the nanoparticles comprise gold. In other embodiments, the nanoparticles comprise iodine.

Gold or iodine nanoparticles also have architectures which provide tunable optical properties. In various embodiments, gold or iodine nanoparticles are configured for optical imaging techniques. For example, the optical and electronic properties can be controlled by controlling the size of the nanoparticle, varying the aspect ratio, or rationally assembling nanoparticles into a specific shape. Those of skill in the art will understand that the size of the gold or iodine nanoparticle can be designed to have specific properties for different applications. For example, the size of the gold or iodine nanoparticle can be designed for colorimeric detection, as described in Martin and Mitchell, Anal. Chem. 1998 pp. 332. Additionally, due to their tunable optical properties, multifunctional polymer modified gold or iodine offer ultrasensitive surface-enhanced Raman detection of biomolecules such as DNA and cancer markers. Metallic gold nanoparticles with surface plasmon behavior have been used as unique optical probes for colorimetric sensing and ultrasensitive surface-enhanced Raman detection of biomolecules such as DNA and cancer markers. Cheon, J. and Lee, Jae-Hyun, Synergistically Integrated Nanoparticles as Multimodal Probes for Nanobiotechnology, Accounts of Chem. Res., published online at www.pubs.acs.org/acr on Aug. 13, 2008.

Gold or iodine nanoparticles may be prepared by methods known in the art, including those disclosed by Burda et al., Chem. Rev. 2005, 105, 1025-1102 and Daniel and Astruc Chem. Rev. 2004, 104, 293-346. Growth methods, including the template, electrochemical, or seeded growth methods, are disclosed by Pérez-Juste et al., Coordination Chemistry Reviews 249 (2005) 1870-1901. Seed particle methods are further described in Murphy et al. J. Phys. Chem. B 2005, 109, 13857-13870. Gold or iodine nanoparticles can also be prepared to have specific surface structures by citrate reduction, two phage synthesis and thiol stabilization, sulfur stabilization, and stabilization with other ligands as described by Daniel and Astruc, Chem. Rev. 2004, 104, 293-346.

### Gadolinium Metal Organic Framework

Gadolinium (III) (Gd³⁺) functions as a MRI contrast agent (CA). Gadolinium enhances the image contrast by increasing water proton relaxation rates. When conjugated to targeting agents, gadolinium metal organic frameworks (MOFs) are effective site-specific MRI contrast agents owing to their large metal payload.

“Gadolinium metal organic framework” as used herein refers to a MOF containing gadolinium (III). Gadolinium MOFs include, but are not limited to, gadolinium (III) MOFs such as those containing carboxylic acids, ligands, and polymers. Representative examples of gadolinium (III) MOFs include Gd(1,4-benzenedicarboxylate)1.5(H₂O)₂ (also known as Gd(1,4-BDC)1.5(H₂O)₂), Gd₂O₃, gadolinium nitrate and emulsions thereof, and gadolinium fluoride.

Gadolinium MOFs can be synthesized by any method known in the art. In a certain embodiment, the gadolinium particles are synthesized as described in, e.g., Rieter, W. J.; et al. J. Am. Chem. Soc. 2006, 128, 9024-9025. The quantity of gadolinium (III) in gadolinium MOFs can be controlled as described in the art by controlling reaction conditions. Rieter, W. J. et al., J. Am. Chem. Soc. 2006 128, 9024-9025.

In alternative embodiments, gadolinium nanoparticles can be synthesized to have a shell morphology with a functionalized polymer on the surface. For example, gadolinium nanoparticles having a paramagnetic Gd₂O₃ core can be produced by encapsulating Gd₂O₃ cores within a polysiloxane shell which carries organic fluorophores and carboxylated PEG covalently tethered to the inorganic network as described in Bridot et al., J. Am. Chem. Soc.; (Article); 2007; 129(16); 5076-5084. In other embodiments, the gadolinium nanoparticles can be synthesized as inorganic/organic hybrid molecules, as described in Hifumi et al. J. Am. Chem. Soc., 128 (47), 15090-15091, 2006.

The quantity of gadolinium in a gadolinium MOF can be modified to adjust contrast in MRI techniques. The quantity of gadolinium nanoparticles can be adjusted as, for example, in Ahrens et al., Proc. Nat'l. Acad. Sci. 95(15) 8443-8448 (1998). Various parameters of gadolinium nanoparticles can be modified. Parameters include the concentration of gadolinium (III), size, aspect ratio, and surface-to-volume concentration of gadolinium (III) in the nanoparticle as described in e.g. Rieter, W. J. et al., J. Am. Chem. Soc. 2006 128, 9024-9025.

### Gadolinium Coated X-ray Computed Tomography Contrast Agent Nanoparticles

A gadolinium MOF can be disposed on a computed tomography contrast agent nanoparticle, which may be a gold or iodine nanoparticle. The MOF may be attached to the gold or iodine nanoparticle via covalent, noncovalent, ionic, Van der Waals, or other types of bonds.

A reverse microemulsion reaction is used to coat gold or iodine nanoparticles with gadolinium MOFs. In one embodiment two aqueous solutions are prepared, one of GdCl₃ (0.5M) and the other containing 1,4-benzenedicarboxylic acid (1,4-BDC) (0.075M). Next, the GdCl₃ and 1,4-BDC aqueous solutions are combined into a heptane/hexanol/cetyltrimethylammonium bromide (0.05M) microemulsion. Gold or iodine nanoparticles are then added to the solution and the reaction is stirred vigorously for 24 hours at room temperature. The nanoparticles are washed several times in ethanol and finally stored in water.

A polymer may be affixed to the MOF. The polymer coating may be created either by polymerizing monomers from the surface of the MOF, or by first polymerizing monomers and then attaching the resulting polymers to the MOF (FIG. 1).

The polymer coating may be added directly to the MOF via a polymer precursor, or through an initiator, formed by creating imperfections on the MOF, as described immediately below.

### Forming Initiators on the Nanoparticle Surface

Prior to growing polymers on the surface of gadolinium coated computed tomography contrast agent nanoparticles, the nanoparticle can be treated to form imperfections (or initiators) on the gadolinium MOF surface. In various embodiments, initiators facilitate polymer formation or polymer precursor binding.

### Polymerization

Polymerization can be performed by any method known in the art. Polymerization methods that can be used are described in Principles of Polymerization, 4th Edition (2004) by George Odian, Published by Wiley-Interscience, which is incorporated herein by reference in its entirety. Various methods of polymerization include reversible addition-fragmentation chain transfer (RAFT), Atom Transfer Radical Polymerization (ATRP), Stable Free Radical Polymerization (SFRP), and conventional free radical polymerization.

RAFT polymerization operates on the principle of degenerative chain transfer. Without being limited to a particular mechanism, Scheme 1 shows a proposed mechanism for RAFT polymerization. In Scheme 1, RAFT polymerization involves a single- or multi-functional chain transfer agent (CTA), such as the compound of formula (I), including dithioesters, trithiocarbonates, xanthates, and dithiocarbamates. The initiator produces a free radical, which subsequently reacts with a polymerizable monomer. The monomer radical reacts with other monomers and propagates to form a chain, Pn*, which can react with the CTA. The CTA can fragment, either forming R*, which will react with another monomer that will form a new chain Pm*, or Pn*, which will continue to propagate. In theory, propagation to the Pm* and Pn* will continue until no monomer is left or a termination step occurs. In particular circumstances, after the first polymerization has finished, a second monomer can be added to the system to form a block copolymer.

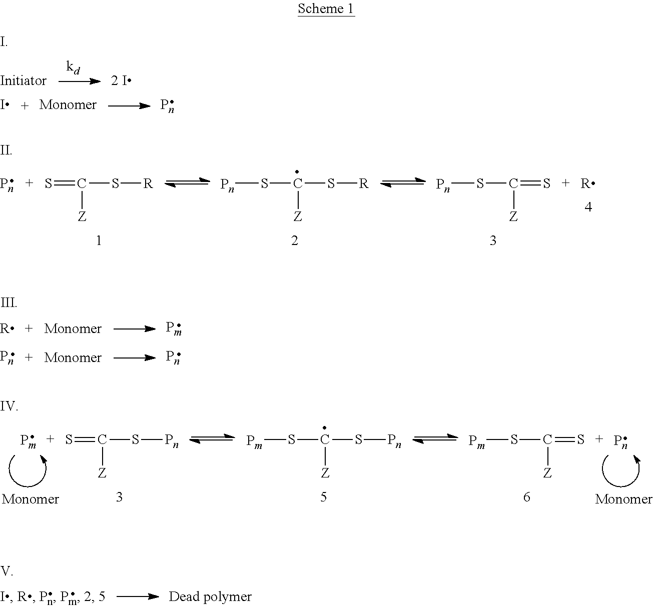

RAFT polymerization involves a similar mechanism as traditional free radical polymerization systems, with the difference of a purposely added CTA. Addition of a growing chain to a macro-CTA yields an intermediate radical, which can fragment to either the initial reactants or a new active chain. With a high chain transfer constant and the addition of a high concentration of CTA relative to conventional initiator, synthesis of a polymer with a high degree of chain-end functionality and with well defined molecular weight properties is obtained.

In particular embodiments, RAFT polymerization is used to produce a variety of well-defined, novel polymers that are either polymerized from the surface of the MOF, or are polymerized and then attached to the MOF. RAFT polymerization shows great promise in the synthesis of multifunctional polymers due to the versatility of monomer selection and polymerization conditions, along with the ability to produce well-defined, narrow polydispersity polymers with both simple and complex architectures.

In particular embodiments, RAFT polymerization is used to produce a variety of well-defined, novel biocopolymers as constructs for multifunctional systems for the surface modification of nanoparticles comprising a computed tomography contrast agent nanoparticle, such as a gold or iodine nanoparticle, coated in a MOF of gadolinium. The inherent flexibility of RAFT polymerizations makes it a candidate to produce well-defined polymer structures with a high degree of functionality capable of providing increased therapeutic/targeting agent loading and loading efficiency. For example, RAFT can be successfully used to produce well-defined activated biocopolymer constructs with N-acryloxysuccinimide (NAOS) pendant functionalities. The succinimide side groups have allowed covalent conjugation of bioactive agents such as fluorescent tags, nucleotides, peptides, and antibodies. R. P. Sebra, Langmuir 2005, 21, 10907-10911; M. J. Yanjarappa, Biomacromolecules 2006, 7, 1665-1670. Incorporation of NAOS into copolymers provides a route of manipulating loading efficiency and stability of bioactive agents. Additional tailoring of the copolymer conjugate system with tumor targeting or therapeutic agents allows specific localization and treatment to be achieved, thereby increasing in vivo performance.

Polymers synthesized by RAFT include chain transfer agents (CTAs). As used herein, a RAFT chain transfer agent is defined as having the chemical structure of Formula (IV):

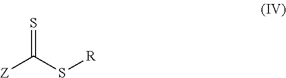

CTAs agents possessing the thiocarbonylthio moiety, impart reactivity to free-radical polymerization due to the facile nature of radical addition to C═S bonds which contributes to faster chain equilibration in the chain transfer step. The transfer constants of RAFT CTAs depend on the Z and R substituents. In certain embodiments, the Z group is a free radical stabilizing species to ensure rapid addition across the C═S bond.

In certain embodiments, the R group is chosen so that it possesses an equal or greater ability to leave as compared to the addition species. It is also of importance that the R group be able to reinitiate the polymerization after fragmentation. In certain embodiments, R can fragment from the intermediate quickly and is able to re-initiate polymerization effectively.

Exemplary CTAs include, but are not limited to, cumyl dithiobenzoate (CDTB) and S-1-Dodecyl-S′-(α,α′-dimethyl-α″-acetic acid) trithiocarbonate (DATC).

### Grafting Polymers and Polymer Precursors to Nanoparticles

In certain aspects, polymers can be grafted to the gadolinium MOF after polymerization. Selecting a CTA structure of formula (I) allows for control of the polymerization. The Z group activates the thio-carbonyl (C═S) group for radical addition and allows for the radical intermediate to be stabilized in the transition state.

Schemes 2 and 3 show grafting trithiocarbonate and dithioester RAFT agents to the surface of a gadolinium nanoparticle. Scheme 2 shows a first step of RAFT polymerization of the alkene in the presence of the trithiocarbonate, and Scheme 3 shows a first step of RAFT polymerization of the alkene in the presence of the dithioester.

The RAFT polymer is grafted to the surface of the nanoparticle. Without being limited to any particular mechanism, the nanoparticle is covalently grafted to the nanoparticle surface. The reduced polymer is covalently grafted to the nanoparticle.

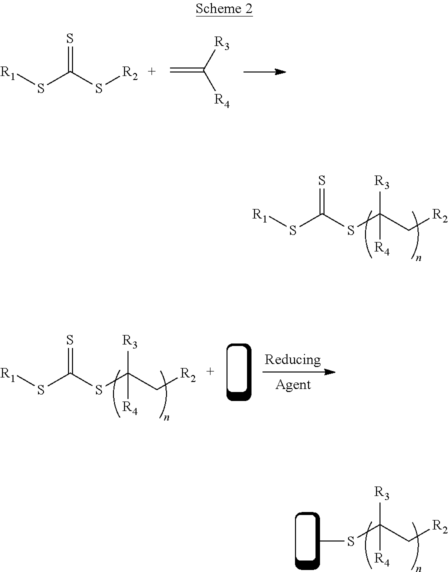

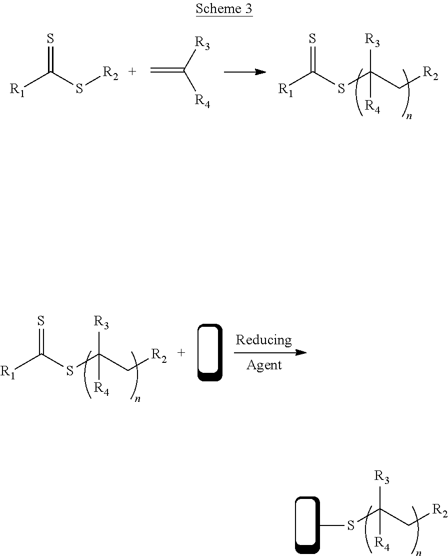

In Schemes 2 and 3, R₁, R₂, R₃, and R₄ are each independently selected from hydrogen, alkyl, substituted alkyl, alkoxy, substituted alkoxy, acyl, substituted acyl, acylamino, substituted acylamino, alkylamino, substituted alkylamino, alkylsulfinyl, substituted alkylsulfinyl, alkylsulfonyl, substituted alkylsulfonyl, alkylthio, substituted alkylthio, alkoxycarbonyl, substituted alkoxycarbonyl, aryl, substituted aryl, arylalkyl, substituted arylalkyl, aryloxy, substituted aryloxy, aryloxycarbonyl, substituted aryloxycarbonyl, carbamoyl, substituted carbamoyl, cycloalkyl, substituted cycloalkyl, cycloheteroalkyl, substituted cycloheteroalkyl, dialkylamino, substituted dialkylamino, halo, heteroalkyl, substituted heteroalkyl, heteroaryl, substituted heteroaryl, heteroarylalkyl, substituted heteroarylalkyl, heteroalkyloxy, substituted heteroalkyloxy, heteroaryloxy, and substituted heteroaryloxy.

A specific example of RAFT polymers attached to the surface of the gadolinium particle after polymerization is depicted in Scheme 4 below. Scheme 4 depicts a method of attaching a modified polymer to a gadolinium nanoparticle to produce a gadolinium nanoparticle conjugate. The modified polymer can be added to the gadolinium nanoparticle after reduction of the trithiocarbonate group to a thiol to produce a gadolinium nanoparticle conjugate.

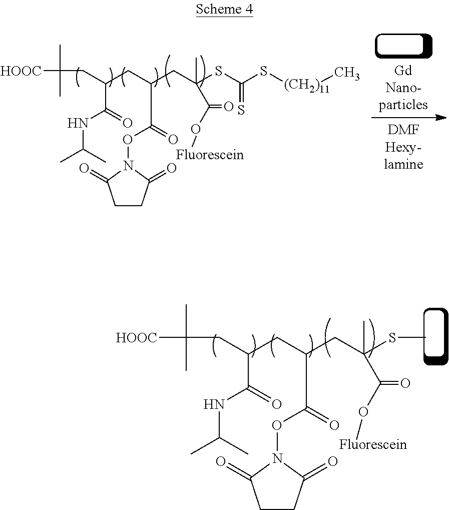

### Grafting From Nanoparticles

In the two examples of generalized RAFT polymerization described above in Schemes 2 and 3, as well as the specific example in Scheme 4, polymerization occurs prior to grafting to the gadolinium nanoparticle surfaces (i.e. “grafting to” the nanoparticle surface).

Alternatively, the RAFT polymerization may be accomplished after grafting a polymer precursor, initiator, or CTA to the nanoparticle surface. Scheme 5 depicts attachment of a CTA to a surface-bound RAFT polymerization. In brief, a polymer precursor is grafted to the surface of the nanoparticle. A CTA is attached to the terminus of the polymer precursor in Step 1. RAFT polymerization is then accomplished in Step 2 directly from the surface of the gadolinium nanoparticle, as described, for example, in Rowe-Konopacki, M. D. and Boyes, S. G. Synthesis of Surface Initiated Diblock Copolymer Brushes from Flat Silicon Substrates Utilizing the RAFT Polymerization Technique. Macromolecules, 40 (4) 879-888, 2007, and Rowe, M. D.; Hammer, B. A. G.; Boyes, S. G. Synthesis of Surface-Initiated Stimuli-Responsive Diblock Copolymer Brushes Utilizing a Combination of ATRP and RAFT Polymerization Techniques. Macromolecules, 41 (12), 4147-4157, 2008.

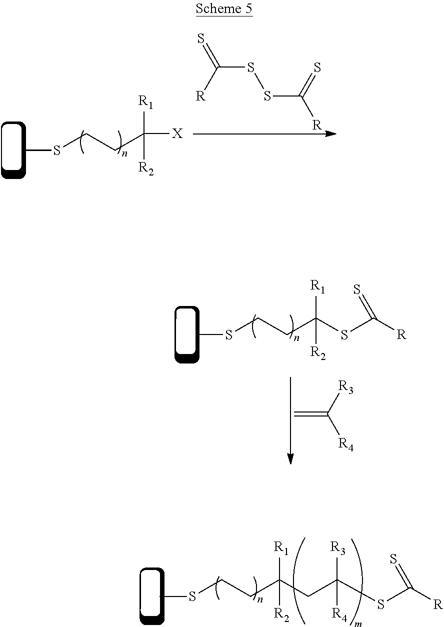

In Scheme 5, n is an integer, and X, R, R₁, R₂, R₃, and R₄ are each independently selected from hydrogen, alkyl, substituted alkyl, alkoxy, substituted alkoxy, acyl, substituted acyl, acylamino, substituted acylamino, alkylamino, substituted alkylamino, alkylsulfinyl, substituted alkylsulfinyl, alkylsulfonyl, substituted alkylsulfonyl, alkylthio, substituted alkylthio, alkoxycarbonyl, substituted alkoxycarbonyl, aryl, substituted aryl, arylalkyl, substituted arylalkyl, aryloxy, substituted aryloxy, carbamoyl, substituted carbamoyl, cycloalkyl, substituted cycloalkyl, cycloheteroalkyl, substituted cycloheteroalkyl, dialkylamino, substituted dialkylamino, halo, heteroalkyl, substituted heteroalkyl, heteroaryl, substituted heteroaryl, heteroarylalkyl, substituted heteroarylalkyl, heteroalkyloxy, substituted heteroalkyloxy, heteroaryloxy, and substituted heteroaryloxy. In certain embodiments, X is a halide such as fluorine, bromine, chlorine and iodine. A specific example of the reaction of Scheme 5 is depicted in Scheme 6.

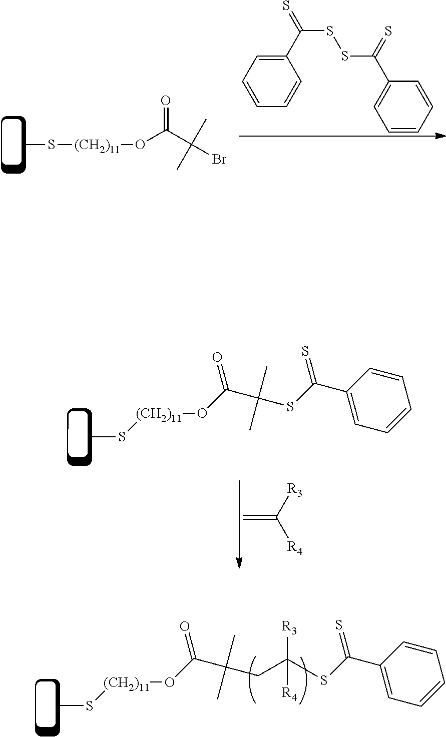

R₃ and R₄ are each independently selected from hydrogen, alkyl, substituted alkyl, alkoxy, substituted alkoxy, acyl, substituted acyl, acylamino, substituted acylamino, alkylamino, substituted alkylamino, alkylsulfinyl, substituted alkylsulfinyl, alkylsulfonyl, substituted alkylsulfonyl, alkylthio, substituted alkylthio, alkoxycarbonyl, substituted alkoxycarbonyl, aryl, substituted aryl, arylalkyl, substituted arylalkyl, aryloxy, substituted aryloxy, aryloxycarbonyl, substituted aryloxycarbonyl, carbamoyl, substituted carbamoyl, cycloalkyl, substituted cycloalkyl, cycloheteroalkyl, substituted cycloheteroalkyl, dialkylamino, substituted dialkylamino, halo, heteroalkyl, substituted heteroalkyl, heteroaryl, substituted heteroaryl, heteroarylalkyl, substituted heteroarylalkyl, heteroalkyloxy, substituted heteroalkyloxy, heteroaryloxy, and substituted heteroaryloxy.

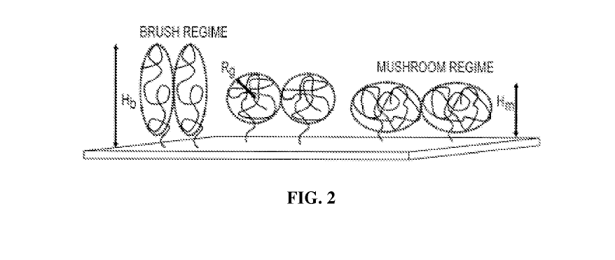

Grafting from the surface of the nanoparticle as depicted above allows formation of a “brush” configuration of polymers. With reference to FIG. 2, polymers attached to a gadolinium surface can be spaced differently on a surface. Without wishing to be held to a specific theory or mechanism of action, the accessibility of the therapeutic agents and targeting agents to the surrounding environment can be at least partially controlled by how closely together the polymers are spaced on the surface of the nanoparticle. When the distance between the polymers is greater than the length of the polymer, the polymers adopt a “mushroom” configuration in which the entirety of the polymer can be accessible to surrounding environment, including the binding site of a targeting agent or therapeutic agent attached to the polymer. Conversely, when the distance between the polymer chains is shorter than the attached polymer, the polymers have a brush conformation, in which the terminal portions of the polymer are accessible to the surrounding environment. If the therapeutic and/or targeting agents are attached to the terminus of the polymers arranged in a “brush” conformation, then the therapeutic and/or targeting agents can be accessible to the surrounding environment.

Desired polymer configuration on the surface can be achieved by growing the polymers from the surface of the nanoparticle. In certain aspects, the polymerization is initiated directly from substrate via immobilized initiators. The brush polymer conformation can be achieved by forming the polymer from the nanoparticle surface, or alternatively by utilizing separately or combining atom transfer radical polymerization (ATRP) and RAFT polymerization. Growing the polymers from the surface allows immobilized polymerization initiators to be tailored for a wide range of polymerization techniques and substrates.

In particular, synthesizing polymer brushes requires control of the polymer molecular weight (i.e. brush thickness), narrow polydispersities, and control of the composition. In the two examples of generalized RAFT polymerization described above in Schemes 5 and 6, polymerization occurs prior to grafting to the gadolinium nanoparticle surfaces (i.e. “grafting to” the nanoparticle surface).

### Functional Groups

Functional groups are groups that can be covalently linked to the polymer and/or covalently linked to the therapeutic agents or pH responsive polymers, and/or bonded to the nanoparticles. The functional groups include any group that can be reacted with another compound to form a covalent linkage between the compound and the polymer extending from the nanoparticle. Exemplary functional groups can include carboxylic acids and carboxylic acid salt derivatives, acid halides, sulfonic acids and sulfonic acid salts, anhydride derivatives, hydroxyl derivatives, amine and amide derivatives, silane derivations, phosphate derivatives, nitro derivatives, succinimide and sulfo-containing succinimide derivatives, halide derivatives, alkene derivatives, morpholine derivatives, cyano derivatives, epoxide derivatives, ester derivatives, carbazole derivatives, azide derivatives, alkyne derivatives, acid containing sugar derivatives, glycerol analogue derivatives, maleimide derivatives, protected acids and alcohols, and acid halide derivatives. The functional groups can be substituted or unsubstituted, as described herein.

Functional groups can be attached to the polymer during polymerization as depicted herein.

Alternatively, functional groups can be attached to the polymer backbone via a linker. The term “linker” as used herein refers to any chemical structure that can be placed between the polymer and functional group. For example, linkers include a group including alkyl, substituted alkyl, alkoxy, substituted alkoxy, acyl, substituted acyl, acylamino, substituted acylamino, alkylamino, substituted alkylamino, alkylsulfinyl, substituted alkylsulfinyl, alkylsulfonyl, substituted alkylsulfonyl, alkylthio, substituted alkylthio, alkoxycarbonyl, substituted alkoxycarbonyl, aryl, substituted aryl, arylalkyl, substituted arylalkyl, aryloxy, substituted aryloxy, carbamoyl, substituted carbamoyl, cycloalkyl, substituted cycloalkyl, cycloheteroalkyl, substituted cycloheteroalkyl, dialkylamino, substituted dialkylamino, halo, heteroalkyl, substituted heteroalkyl, heteroaryl, substituted heteroaryl, heteroarylalkyl, substituted heteroarylalkyl, heteroalkyloxy, substituted heteroalkyloxy, heteroaryloxy, and substituted heteroaryloxyalkyl groups. In various non-limited exemplary embodiments, the groups can be from C₁ to C₁₀, C₂₀, or C₃₀.

In various embodiments, the linker can include a conjugated bond, such as acetylene (—C═C—, also called alkyne or ethyne), alkene (—CH═CH—, also called ethylene), substituted alkene (—CR═CR—, —CH═CR— and —CR═CH—), amide (—NH—CO— and —NR—CO— or —CO—NH— and —CO—NR—), azo (—N═N—), esters and thioesters (—CO—O—, —O—CO—, —CS—O— and —O—CS—) and other conjugated bonds such as (—CH═N—, —CR═N—, —N═CH— and —N═CR—), (—SiH═SiH—, —SiR═SiH—, —SiR═SiH—, and —SiR═SiR—), and (—SiH═CH—, —SiR═CH—, —SiH═CR—, —SiR═CR—, —CH═SiH—, —CR═SiH—, —CH═SiR—, and —CR═SiR—). Particularly certain bonds are acetylene, alkene, amide, and substituted derivatives of these three, and azo. The linker can also be carbonyl, or a heteroatom moiety, wherein the heteroatom is selected from oxygen, sulfur, nitrogen, silicon, or phosphorus. Thus, suitable heteroatom moieties include, but are not limited to, —NH and —NR, wherein R is as defined herein; substituted sulfur; sulfonyl (—SO₂—) sulfoxide (—SO—); phosphine oxide (—PO— and —RPO—); and thiophosphine (—PS— and —RPS—). The linker can also be a peptidyl spacer such as Gly-Phe-Leu-Gly.

### Targeting Agents and pH Responsive Polymers

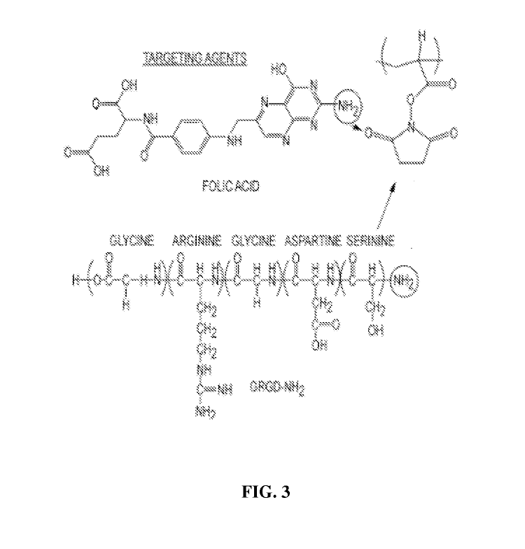

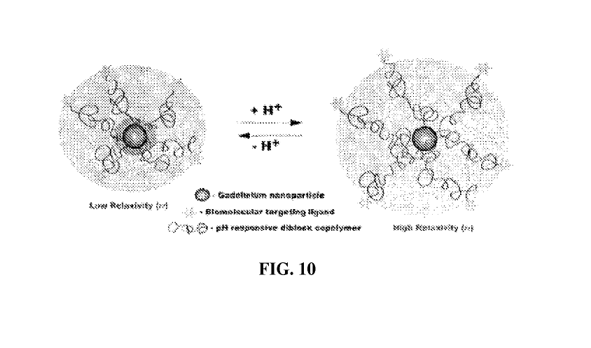

Targeting agents and pH responsive polymers can be covalently attached to the polymer. FIG. 3 shows an example of targeting molecules (folic acid or an RGD sequence) binding to a functionalized polymer grafted to a nanoparticle. The functional groups attach to the polymer backbone by reaction with the succinimide functional group. FIG. 10 depicts an example of a pH responsive polymer bound to a gadolinium MOF nanoparticle.

In various embodiments, the succinimide group of PNAOS provides an attachment point for a variety of pH responsive polymers and targeting agents, such as, but not limited to, folic acid, and GRGD sequences, through pre- and post-polymerization modification. Unreacted succinimide groups can further be converted to non-bioactive groups to reduce in vivo side reactions. Conjugation of targeting and imaging agents and pH responsive polymers to the copolymer provides a multifaceted system, which has potential in decreasing toxicity through directed targeting with the ability to image through optical, magnetic resonance, or computed tomography.

It will be understood by those of skill in the art that various targeting agents or pH responsive polymers can be selected for attachment to functional groups. Further, it will be understood that a linker can be placed between the functional groups and the targeting agents and pH responsive polymers. The linker can be cleavable or non-cleavable. In certain instances, pH responsive polymers can be non-cleavable.

pH responsive polymers are compounds that change in some way in response to pH and/or changes in pH, such as extracellular pH (pHe). A pH responsive polymer may be a diblock copolymer as shown in FIG. 10. Without being limited to any mechanism or mode of action, when a pH responsive polymer is in an open or extended conformation, it allows water to access the gadolinium, which increases the relaxivity (r₁) of the nanoparticle. In the open or extended conformation, the MRI signal produced by the nanoparticle may be turned on. When a pH responsive polymer is in a closed or collapsed conformation, it limits or prevents water from accessing the gadolinium, which decreases the relaxivity (r₁) of the nanoparticle. In the closed or collapsed conformation, the MRI signal produced by the nanoparticle may be turned off. In one embodiment, a pH responsive polymer assumes an open or extended conformation under acidic conditions, or upon a decrease in pHe, and assumes a closed or collapsed conformation under neutral or basic conditions, or upon an increase in pHe. In another embodiment, a pH responsive polymer assumes an open or extended conformation under neutral or basic conditions, or upon an increase in pHe, and assumes a closed or collapsed conformation under acidic conditions, or upon a decrease in pHe.

Targeting agents are compounds with a specific affinity for a target compound, such as a cell surface epitope associated with a specific disease state. Targeting agents may be attached to a nanoparticle surface to allow targeting of the nanoparticle to a specific target. Non-limiting examples of targeting agents include an amino acid sequence including the RGD peptide, an NGR peptide, folate, Transferrin, GM-CSF, Galactosamine, peptide linkers including growth factor receptors (e.g. IGF-1R, MET, EGFR), antibodies and antibody fragments including anti-VEGFR, Anti-ERBB2, Anti-tenascin, Anti-CEA, Anti-MUC1, and Anti-TAG72, mutagenic bacterial strains, and fatty acids.

Targeting agents can include any number of compounds known in the art. In certain situations, the targeting agent specifically binds to a particular biological target. Non-limiting examples of biological targets include tumor cells, bacteria, viruses, cell surface proteins, cell surface receptors, cell surface polysaccharides, extracellular matrix proteins, intracellular proteins, and intracellular nucleic acids.

In various embodiments, targeting agents can be chosen for the different ways in which they interact with tumors. For example, when the targeting agent folic acid is taken into the cells by the folate receptors, RGD receptors are expressed on the surface of the cells, resulting in the nanostructures localizing to the cell surface. The folate receptor is known to be over expressed in cancer cells in the case of epithelial malignancies, such as ovarian, colorectal, and breast cancer, whereas in most normal tissue it is expressed in very low levels.

The nanoparticles and methods described herein are not limited to any particular targeting agent, and a variety of targeting agents can be used. The targeting agents can be, for example, various specific ligands, such as antibodies, monoclonal antibodies and their fragments, folate, mannose, galactose and other mono-, di-, and oligosaccharides, and RGD peptide. Examples of such targeting agents include, but are not limited to, nucleic acids (e.g., RNA and DNA), polypeptides (e.g., receptor ligands, signal peptides, avidin, Protein A, and antigen binding proteins), polysaccharides, biotin, hydrophobic groups, hydrophilic groups, drugs, and any organic molecules that bind to receptors. In some instances, a nanoparticle described herein can be conjugated to one, two, or more of a variety of targeting agents. For example, when two or more targeting agents are used, the targeting agents can be similar or dissimilar. Utilization of more than one targeting agent in a particular nanoparticle can allow the targeting of multiple biological targets or can increase the affinity for a particular target.

In some instances, the targeting agents are antigen binding proteins or antibodies or binding portions thereof. Antibodies can be generated to allow for the specific targeting of antigens or immunogens (e.g., tumor, tissue, or pathogen specific antigens) on various biological targets (e.g., pathogens, tumor cells, normal tissue). Such antibodies include, but are not limited to, polyclonal antibodies; monoclonal antibodies or antigen binding fragments thereof; modified antibodies such as chimeric antibodies, reshaped antibodies, humanized antibodies, or fragments thereof (e.g., Fv, Fab′, Fab, F(ab′)2); or biosynthetic antibodies, e.g., single chain antibodies, single domain antibodies (DAB), Fvs, or single chain Fvs (scFv).

Methods of making and using polyclonal and monoclonal antibodies are well known in the art, e.g., in Harlow et al, Using Antibodies: A Laboratory Manual: Portable Protocol I. Cold Spring Harbor Laboratory (Dec. 1, 1998). Methods for making modified antibodies and antibody fragments (e g , chimeric antibodies, reshaped antibodies, humanized antibodies, or fragments thereof, e.g., Fab′, Fab, F(ab′)2 fragments); or biosynthetic antibodies (e.g., single chain antibodies, single domain antibodies (DABs), Fv, single chain Fv (scFv), and the like), are known in the art and can be found, e.g. , in Zola, Monoclonal Antibodies: Preparation and Use of Monoclonal Antibodies and Engineered Antibody Derivatives, Springer Verlag (Dec. 15, 2000; 1st edition). In some instances, the antibodies recognize tumor specific epitopes (e.g., TAG-72 (Kjeldsen et al, Cancer Res., 48:2214-2220 (1988); U.S. Pat. Nos. 5,892,020; 5,892,019; and 5,512,443); human carcinoma antigen (U.S. Pat. Nos. 5,693,763; 5,545,530; and 5,808,005); TP1 and TP3 antigens from osteocarcinoma cells (U.S. Pat. No. 5,855,866); Thomsen-Friedenreich (TF) antigen from adenocarcinoma cells (U.S. Pat. No. 5,110,911); “KC-4 antigen” from human prostrate adenocarcinoma (U.S. Pat. Nos. 4,708,930 and 4,743,543); a human colorectal cancer antigen (U.S. Pat. No. 4,921,789); CA125 antigen from cystadenocarcinoma (U.S. Pat. No. 4,921,790); DF3 antigen from human breast carcinoma (U.S. Pat. Nos. 4,963,484 and 5,053,489); a human breast tumor antigen (U.S. Pat. No. 4,939,240); p97 antigen of human melanoma (U.S. Pat. No. 4,918,164); carcinoma or orosomucoid-related antigen (CORA) (U.S. Pat. No. 4,914,021); a human pulmonary carcinoma antigen that reacts with human squamous cell lung carcinoma but not with human small cell lung carcinoma (U.S. Pat. No. 4,892,935); T and Tn haptens in glycoproteins of human breast carcinoma (Springer et ah, Carbohydr. Res., 178:271-292 (1988)), MSA breast carcinoma glycoprotein (Tjandra et al, Br. J. Surg., 75:811-817 (1988)); MFGM breast carcinoma antigen (Ishida et al, Tumor Biol, 10: 12-22 (1989)); DU-PAN-2 pancreatic carcinoma antigen (Lan et al, Cancer Res., 45:305-310 (1985)); CA125 ovarian carcinoma antigen (Hanisch et ah, Carbohydr. Res., 178:29-47 (1988)); and YH206 lung carcinoma antigen (Hinoda et al, Cancer J., 42:653-658 (1988)). For example, to target breast cancer cells, the nanoparticles can be modified with folic acid, EGF, FGF, and antibodies (or antibody fragments) to the tumor-associated antigens MUC 1, cMet receptor and CD56 (NCAM).

Other antibodies that can be used recognize specific pathogens (e.g., Legionella peomophilia, Mycobacterium tuberculosis, Clostridium tetani, Hemophilus influenzae, Neisseria gonorrhoeae, Treponema pallidum, Bacillus anthracis, Vibrio cholerae, Borrelia burgdorferi, Cornebacterium diphtheria, Staphylococcus aureus, human papilloma virus, human immunodeficiency virus, rubella virus, and polio virus).

In some instances, the targeting agents include a signal peptide. These peptides can be chemically synthesized or cloned, and expressed and purified using known techniques. Signal peptides can be used to target the nanoparticles described herein to a discreet region within a cell. In some situations, specific amino acid sequences are responsible for targeting the nanoparticles into cellular organelles and compartments. For example, the signal peptides can direct a nanoparticle described herein into mitochondria. In other examples, a nuclear localization signal is used.

In other instances, the targeting agent is a nucleic acid (e.g., RNA or DNA). In some examples, the nucleic acid targeting agents are designed to hybridize by base pairing to a particular nucleic acid (e.g., chromosomal DNA, mRNA, or ribosomal RNA). In other situations, the nucleic acids bind a ligand or biological target. For example, the nucleic acid can bind reverse transcriptase, Rev or Tat proteins of HIV (Tuerk et al, Gene, 137(1):33-9 (1993)); human nerve growth factor (Binkley et al, Nuc. Acids Res., 23(16):3198-205 (1995)); or vascular endothelial growth factor (Jellinek et al, Biochem., 83(34): 10450-6 (1994)). Nucleic acids that bind ligands can be identified by known methods, such as the SELEX procedure (see, e.g., U.S. Pat. Nos. 5,475,096; 5,270,163; and 5,475,096; and WO 97/38134; WO 98/33941; and WO 99/07724). The targeting agents can also be aptamers that bind to particular sequences.

The targeting agents can recognize a variety of epitopes on biological targets (e.g., pathogens, tumor cells, or normal cells). For example, in some instances, the targeting agent can be sialic acid to target HIV (Wies et al , Nature, 333:426 (1988)), influenza (White et al, Cell, 56:725 (1989)), Chlamydia (Infect. Immunol, 57:2378 (1989)), Neisseria meningitidis, Streptococcus suis, Salmonella, mumps, newcastle, reovirus, Sendai virus, and myxovirus; and 9-OAC sialic acid to target coronavirus, encephalomyelitis virus, and rotavirus; non-sialic acid glycoproteins to target cytomegalovirus (Virology, 176:337 (1990)) and measles virus (Virology, 172:386 (1989)); CD4 (Khatzman et al, Nature, 312:763 (1985)), vasoactive intestinal peptide (Sacerdote et al, J. of Neuroscience Research, 18: 102 (1987)), and peptide T (Ruff et al, FEBS Letters, 211 : 17 (1987)) to target HIV; epidermal growth factor to target vaccinia (Epstein et al , Nature, 318: 663 (1985)); acetylcholine receptor to target rabies (Lentz et al, Science 215: 182 (1982)); Cd3 complement receptor to target Epstein-Barr virus (Cara et al, J. Biol. Chem., 265: 12293 (1990)); .beta.-adrenergic receptor to target reovirus (Co et al, Proc. Natl. Acad. ScL USA, 82: 1494 (1985)); ICAM-I (Marlin et al, Nature, 344:70 (1990)), N-CAM, and myelin-associated glycoprotein MAb (Shephey et al, Proc. Natl. Acad. ScL USA, 85:7743 (1988)) to target rhinovirus; polio virus receptor to target polio virus (Mendelsohn et al, Cell, 56:855 (1989)); fibroblast growth factor receptor to target herpes virus (Kaner et al, Science, 248: 1410 (1990)); oligomannose to target Escherichia coli; and ganglioside GMI to target Neisseria meningitides.

In other instances, the targeting agent targets nanoparticles according to the disclosure to factors expressed by oncogenes. These can include, but are not limited to, tyrosine kinases (membrane-associated and cytoplasmic forms), such as members of the Src family; serine/threonine kinases, such as Mos; growth factor and receptors, such as platelet derived growth factor (PDDG), SMALL GTPases (G proteins), including the ras family, cyclin-dependent protein kinases (cdk), members of the myc family members, including c-myc, N-myc, and L-myc, and bc1-2 family members.

In addition, vitamins (both fat soluble and non-fat soluble vitamins) can be used as targeting agents to target biological targets (e.g., cells) that have receptors for, or otherwise take up, vitamins. For example, fat soluble vitamins (such as vitamin D and its analogs, vitamin E, Vitamin A), and water soluble vitamins (such as Vitamin C) can be used as targeting agents.

In some embodiments, antibodies or ligands may be used to aid in site-specific targeting (T. M. Allen, Nat. Rev. Cancer 2, 750 (Oct, 2002), Y. S. Park, Biosci. Rep. 22, 267 (April, 2002)). Antibodies and antibody fragments are as described herein.

The nanoparticles described herein can be used to characterize diseased cells and tissues, such as by measuring extracellular pH. In this regard, diseases correlated with changes in pH, such as from hypoxic conditions or inflammation, are amenable to characterization using the nanoparticles and methods described herein. An exemplary, non-limiting list of diseases that can be characterized with the subject nanoparticles includes cancer, such as breast and prostate cancer, diabetes, pulmonary hypertension, and cardiac disease.

Targeting agents and pH responsive polymers can be covalently attached to the polymer by RAFT synthesis. The targeting agent or pH responsive polymer is configured to be added to the RAFT polymer during polymerization. As such the targeting agent or pH responsive polymer can be linked directly to the RAFT polymer. Those of skill in the art will recognize that a linker can be added between the targeting agent or pH responsive polymer and the polymer.

Alternatively, targeting agents or pH responsive polymers are linked to the polymer via a functional group as described above. Those of skill in the art will recognize that a linker can be added between the targeting agent or pH responsive polymer and the polymer. Targeting agents can also be attached to pH responsive polymers, as shown in FIG. 10.

Multifunctional synthesis of compounds can be accomplished by RAFT polymerization as depicted in the example of Scheme 7.

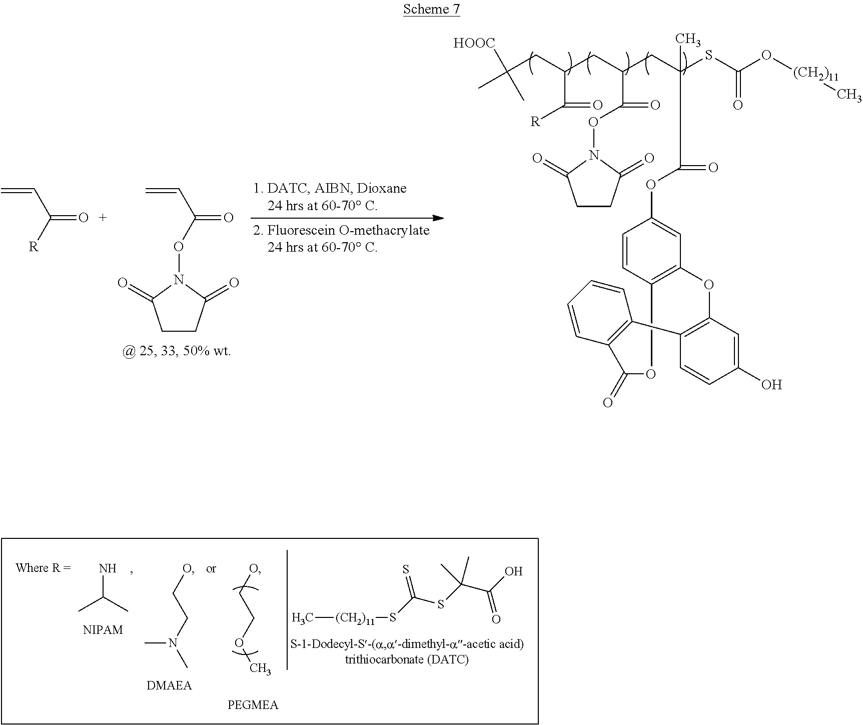

In this embodiment, a succinimide group can be used to attach a functional group to the nanoparticle. An example of biocompatible copolymers containing functional N-acryloyloxysuccinimide (NAOS) monomer units can also be synthesized via RAFT polymerization. A range of copolymer backbones can be used, including, but not limited to, N-isopropylacrylamide (NIPAM), N,N-dimethylaminoethyl acrylate (DMAEA), and poly(ethylene glycol) methyl ether acrylate (PEGMEA). The addition of NAOS into the copolymer backbones has been achieved at a range of weight percents as a means of attachment. The copolymers were synthesized utilizing the well-known trithiocarbonate DATC in dioxane at 60 or 70 degrees, with a fluorescein monomer incorporated near the end of the polymerization. The polymers were characterized via both proton NMR and GPC.

In various embodiments, unreacted succinimide groups can further be converted to non-bioactive groups to reduce in vivo side reactions.

In Scheme 8, a folic acid targeting agent is attached to the succinimide functional group.

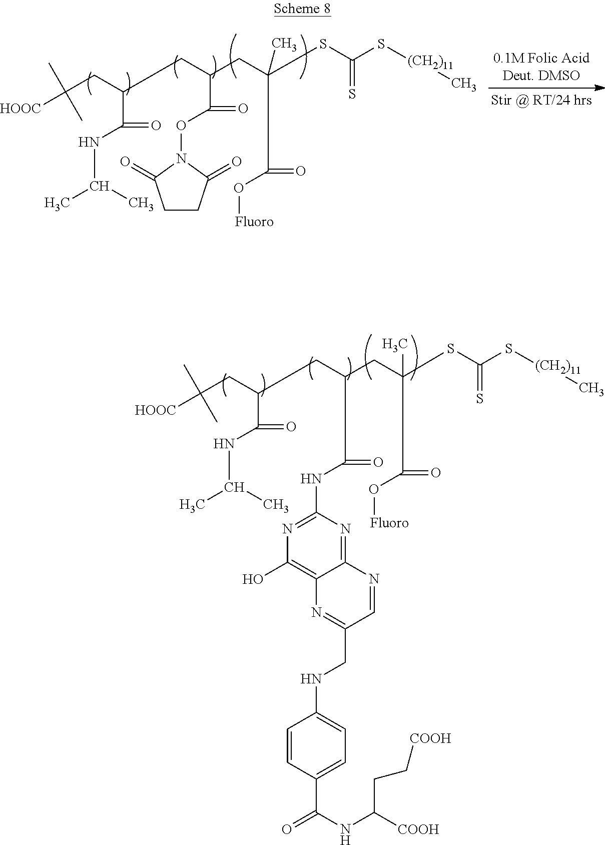

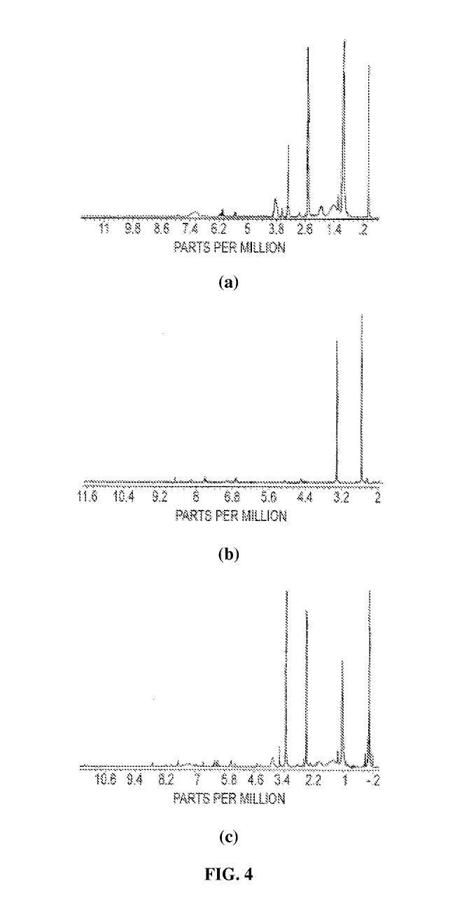

FIG. 4a depicts a ¹H NMR spectrum of PNIPAM-co-PNAOS-co-PFMA copolymer. FIG. 4b depicts a ¹H NMR spectra of folic acid. FIG. 4c depicts a ¹H NMR spectrum of PNIPAM copolymer reacted with folic acid.

### Administration of Nanoparticles

The route and/or mode of administration of a nanoparticle described herein can vary depending upon the desired results.

Methods of administration include, but are not limited to, intradermal, intramuscular, intraperitoneal, intravenous, subcutaneous, intranasal, epidural, oral, sublingual, intracerebral, intravaginal, transdermal, rectal, inhalation, or topical, particularly to the ears, nose, eyes, or skin. The mode of administration is left to the discretion of the practitioner.

In some instances, a nanoparticle described herein is administered locally. This is achieved, for example, by local infusion during surgery, topical application (e.g., in a cream or lotion), by injection, by means of a catheter, by means of a suppository or enema, or by means of an implant, said implant being of a porous, non-porous, or gelatinous material, including membranes, such as sialastic membranes or fibers. In some situations, a nanoparticle described herein is introduced into the central nervous system, circulatory system, or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant.

A nanoparticle described herein is formulated as a pharmaceutical composition that includes a suitable amount of a physiologically acceptable excipient (see, e.g., Remington's Pharmaceutical Sciences pp. 1447-1676 (Alfonso R. Gennaro, ed., 19th ed. 1995)). Such physiologically acceptable excipients can be, e.g., liquids, such as water and oils, including those of petroleum, animal, vegetable, or synthetic origin, such as peanut oil, soybean oil, mineral oil, sesame oil, and the like. The physiologically acceptable excipients can be saline, gum acacia, gelatin, starch paste, talc, keratin, colloidal silica, urea, and the like. In addition, auxiliary, stabilizing, thickening, lubricating, and coloring agents can be used. In one situation, the physiologically acceptable excipients are sterile when administered to an animal. The physiologically acceptable excipient should be stable under the conditions of manufacture and storage and should be preserved against the contaminating action of microorganisms. Water is a particularly useful excipient when a nanoparticle described herein is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. Other examples of suitable physiologically acceptable excipients are described in Remington's Pharmaceutical Sciences pp. 1447-1676 (Alfonso R. Gennaro, ed., 19th ed. 1995). The pharmaceutical compositions, if desired, can also contain minor amounts of wetting or emulsifying agents, or pH buffering agents.

Liquid carriers can be used in preparing solutions, suspensions, emulsions, syrups, and elixirs. A nanoparticle described herein can be suspended in a pharmaceutically acceptable liquid carrier such as water, an organic solvent, a mixture of both, or pharmaceutically acceptable oils or fat. The liquid carriers can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives described herein, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. The liquid carriers can be in sterile liquid form for administration. The liquid carriers for pressurized compositions can be halogenated hydrocarbons or other pharmaceutically acceptable propellants.

In other instances, a nanoparticle described herein is formulated for intravenous administration. Compositions for intravenous administration can comprise a sterile isotonic aqueous buffer. The compositions can also include a solubilizing agent. Compositions for intravenous administration can optionally include a local anesthetic such as lignocaine to lessen pain at the site of the injection. The ingredients can be supplied either separately or mixed together in unit dosage form, for example, as a dry lyophilized powder or water-free concentrate in a hermetically sealed container such as an ampule or sachette indicating the quantity of active agent. Where a nanoparticle described herein is administered by infusion, it can be dispensed, for example, with an infusion bottle containing sterile pharmaceutical grade water or saline. Where a nanoparticle described herein is administered by injection, an ampule of sterile water for injection or saline can be provided so that the ingredients can be mixed prior to administration.

In other circumstances, a nanoparticle described herein can be administered across the surface of the body and the inner linings of the bodily passages, including epithelial and mucosal tissues. Such administrations can be carried out using a nanoparticle described herein in lotions, creams, foams, patches, suspensions, solutions, and suppositories (e.g., rectal or vaginal). In some instances, a transdermal patch can be used that contains a nanoparticle described herein and a carrier that is inert to the nanoparticle described herein, is non-toxic to the skin, and that allows delivery of the agent for systemic absorption into the blood stream via the skin. The carrier can take any number of forms such as creams or ointments, pastes, gels, or occlusive devices. The creams or ointments can be viscous liquid or semisolid emulsions of either the oil-in-water or water-in-oil type. Pastes of absorptive powders dispersed in petroleum or hydrophilic petroleum containing a nanoparticle described herein can also be used. A variety of occlusive devices can be used to release a nanoparticle described herein into the blood stream, such as a semi-permeable membrane covering a reservoir containing the nanoparticle described herein with or without a carrier, or a matrix containing the nanoparticle described herein.

### Imaging Agents

Biomedical imaging and diagnostic techniques such as computed X-ray tomography (CT), magnetic resonance imaging (MRI), optical imaging (OI) and positron emission tomography (PET) are useful in modem clinical settings for the diagnosis of various diseases. While these imaging techniques have been responsible for tremendous advances in clinical diagnosis, each has its own advantages and drawbacks, and no single technique includes all the required capabilities for comprehensive biomedical imaging.

Imaging agents can be covalently attached to the polymer. The imaging agents may be attached to a functionalized group on the polymer backbone grafted to a nanoparticle. The functional groups attach to the polymer backbone by reaction with the succinimide functional group. Those of skill in the art will recognize that many different imaging agents that can be attached to the polymer backbone. Without being limited to specific embodiments, imaging agents can be chosen from the group comprising fluorescence agents, radiological agents, and positron emission agents.

Current MRI techniques employ gadolinium as a contrast agent. In certain embodiments, gadolinium metal is highly toxic to cells. For MRI application, this toxicity has been overcome by utilizing chelates to increase stability and compatibility of the metal ion. However, concerns have arisen with current in vivo use of gadolinium chelates due to non-specific cellular uptake and accumulation within healthy cells. W. A. High, J. Am. Acad. Dermatol. 2006,56, 21-26. Several groups have attempted to overcome these issues by using cascade polymers and dendrimers, however size distribution and spatial loading is poor. Id. Gadolinium oxide nanoparticles have proven to be interesting because of their effectiveness as MRI contrast agents. J. L. Bridot, J. Am. Chem. Soc. 2007. H. Hifumi, J. Am. Chem. Soc. 2006, 128, 15090-15091, W. J. Rieter, J. Am. Chem. Soc. 2006, 128, 9024-9025.M. O. Oyewumi, Journal of Controlled Release 2004, 95, 613-626. Modification of these particles with polymers shows promise as a means to compatiblize the surface of gadolinium nanoparticles for in vivo imaging and to affix moieties that will potentially allow for targeting and treatment of cancer cells through control of nanoparticle-cellular surface interactions. Though recent advances have been made in the synthesis and modification of metal nanoparticles, such as gold nanorods, the modification and characterization of metal oxide frameworks, such as gadolinium oxide nanoparticles, is still limited.

Fluorescence agents can be visualized in the visible or near-visible spectra. Fluorescence agents, such as poly(fluorescein O-methacrylate (PFMA), are induced to emit photons by exciting electrons in the molecules by exposure to light energy, typically violet or ultraviolet light. Radiological agents are molecules possessing radioactive material that can be detected by detection of the radioactive decay. PET agents contain radioactive materials that can be detected by a gamma or PET scanner.

It will be understood by those of skill in the art that various imaging agents can be selected for attachment to functional groups. Further, it will be understood that a linker can be placed between the functional groups and the imaging agents. The linker can be cleavable or non-cleavable. For example, in certain instances imaging agents can be cleavable. In certain instances, imaging agents can be non-cleavable.

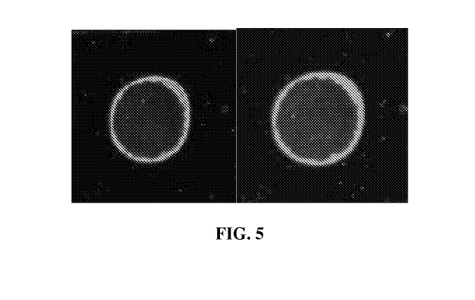

Without being limited by specific embodiments, gadolinium nanoparticles can be modified with a well-defined RAFT copolymer, PNIPAM-co-poly(N-acryloxysuccinmide)(PNAOS)-co-PFMA. Incorporation of the PFMA monomer into the backbone of the copolymer provides a means for measuring polymer incorporation in vitro by fluorescence imaging. In order to confirm the ability of these copolymer modified hybrid nanoparticles to be imaged by fluorescence microscopy, a fluorescence scanner was employed to provide the images of PNIPAM-co-PNAOS-co-PFMA modified gold/gadolinium nanoparticles (FIG. 5). Fluorescence of these particles is readily detectible in copolymer modified gold/lanthanide nanoparticles.

### Multimodal Imaging

In some aspects different imaging agents may be combined to produce a multimodal imaging agent. By way of example but not limitation, multimodal imaging agents may combine MRI and CT, PET and CT, PET and MRI, or MRI and OI. (Bakalova et al, Multimodal Silica Shelled Quantum Dots: Direct Intracellular Delivery, Photosensitization, Toxic, and Microcirculation Effects. Bioconjugate Chem. 19, 1135-1142 (2008); Frullano, L., Meade, T. J. Multimodal MRI Contrast Agents. J. Biol. Inorg. Chem. 12, 939-949 (2007).) In some aspects, these imaging techniques may be complimentary, rather than competitive, and so may aid in minimizing artifacts and enabling precise comparative analysis of images obtained by the different techniques. (Frullano, L., Meade, T. J. Multimodal MRI Contrast Agents. J. Biol. Inorg. Chem. 12, 939-949 (2007)). The presently disclosed nanoparticles can be used a multimodal imaging agents to non-invasively measure pHe and to detect, diagnose, and characterize disease states.

### Gadolinium-Based Imaging

MRI involves measuring the nuclear magnetic resonance (NMR) of water protons in a specimen. This may be performed by placing a subject in a magnetic field that may re-orient protons and measuring the time for the affected protons to relax. Protons in differing chemical environments will exhibit different relaxation times. The observed contrast in MRI essentially depends on factors such as the water proton density, the longitudinal relaxation time (T1), and the transverse relaxation time (T2) of these protons. Contrast agents may be used in MRI to aid in diagnostic imaging.

Gadolinium may be used to enhance MRI. (Aime et al, R₂/R₁ Ratiometric Procedure for a Concentration-Independent, pH-Responsive, Gd(III)-Based MRI Agent. J. Am. Chem. Soc. 128, 11326-11327 (2006); Bridot et al., Hybrid Gadolinium Oxide Nanoparticles: Multimodal Contrast Agents for in Vivo Imaging. J. Am. Chem. Soc. 129, 5076-5084 (2007); Hifumi et al., Gadolinium-Based Hybrid Nanoparticles as a Positive MR Contrast Agent. J. Am. Chem. Soc. 128, 15090-15091 (2006)). MRI agents may work by increasing the contrast (or relaxation rate of protons) between the particular organ or tissue of interest and the surrounding tissues in the body. (Caravan, P., Ellison, J. J., McMurry, T. J., Lauffer, R. B. Gadolinium(III) Chelates as MRI Contrast Agents: Structure, Dynamics, and Application. Chem. Rev. 99, 2293-2352 (1999).) These agents may have a local effect on T1 and T2 relaxation times. In one aspect, relaxivity of water protons may be altered by introducing a high spin paramagnetic metal into the system. For example, gadolinium, may be used to alter proton relaxation times. In some aspects, water molecules bound to gadolinium may relax orders of magnitude faster than free water, resulting in dramatic changes in T1 where gadolinium is present. Gadolinium(III) (Gd³⁺) complexes may have a high longitudinal relaxivity (r₁) and thus have an effect mostly on T1-relaxation times of surrounding water protons (as described in Caravan et al., Gadolinium(III) Chelates as MRI Contrast Agents: Structure, Dynamics, and Application. Chem. Rev. 99, 2293-2352 (1999)). This enhanced T1-relaxation time may lead to an increase in signal intensity in T1-weighted images.

### Gold- and Iodine-Based Imaging

Gold (Au) and iodine (I), because of their high atomic numbers and X-ray absorption coefficients, may be used to in various imaging techniques known in the art. (Kim, D., Park, S., Lee, J. H., Jeong, Y. Y., Jon, S. Antibiofouling Polymer-Coated Gold Nanoparticles as a Contrast Agent for in Vivo X-ray Computed Tomography Imaging. J. Am. Chem. Soc. 129, 7661-7665 (2007); Su, C.-H., Sheu, H.-S., Lin, C.-Y., Huang, C.-C., Lo, Y.-W., Pu, Y.-C., Weng, J.-C., Shieh, D.-B., Chen, J.-H., Yeh, C.-S. Nanoshell Magnetic Resonance Imaging Contrast Agents. J. Am. Chem. Soc. 129, 2139-2146 (2007); Huang, X., El-Sayed, I. H., Qian, W., El-Sayed, M. A. Cancer Cell Imaging and Photothermal Therapy in the Near-Infrared Region by Using Gold Nanorods. J. Am. Chem. Soc. 128, 2115-2120 (2006)). By way of example but not limitation, gold or iodine nanoparticles may be used to enhance images produced by CT (as described in Kim et al, Antibiofouling Polymer-Coated Gold Nanoparticles as a Contrast Agent for in Vivo X-ray Computed Tomography Imaging. J. Am. Chem. Soc. 129, 7661-7665 (2007)), and dark field and confocal microscopy (as described in Huang, X., El-Sayed, I. H., Qian, W., El-Sayed, M. A. Cancer Cell Imaging and Photothermal Therapy in the Near-Infrared Region by Using Gold Nanorods. J. Am. Chem. Soc. 128, 2115-2120 (2006)). Dark field and confocal microscopy are optical imaging techniques that may aid visualization by increasing contrast. CT is a non-optical imaging technique that may involve constructing a three dimensional representation of a specimen from a series of two dimensional X-ray images.

In vivo Determination of pH

As described above, relaxivity of a gadolinium MOF nanoparticle may be changed by a pH responsive polymer, such as a pH responsive polymer, attached to the nanoparticle. In some embodiments, under acidic conditions, conformational changes in the pH responsive polymer lead to an increase in relaxivity compared to neutral or basic conditions. In other embodiments, under basic conditions, conformational changes in the pH responsive polymer lead to an increase in relaxivity compared to neutral or acidic conditions.

pHe may be determined in vivo by measuring MRI longitudinal relaxation time of gadolinium MOF nanoparticles modified with pH responsive polymers. The pH responsive polymer represents an improvement over known MRI computed tomographies that suffer from low changes in relaxivity and concomitantly limited sensitivity. As described in Example 3, gadolinium MOF nanoparticles modified with pH responsive polymers via RAFT polymerization demonstrated a 58% change in T1 when the pH was changed from 6 to 3, whereas gadolinium MOF nanoparticles modified with a non-pH responsive polymer demonstrated a 0.8% change in T1 when the pH was changed from 6 to 3. Thus, under acidic conditions, a nanoparticle modified with a pH responsive polymer demonstrated a greater than 70-fold increase in the effect on T1 compared to a non-pH responsive polymer. This effect on T1 is almost an order of magnitude better than recent pH responsive MRI computer tomographies reported in literature, and represents an improvement over known pH responsive MRI computed tomographies.

Measuring the CT signal or image intensity of the presently disclosed nanoparticles in conjunction with MRI can help to accurately measure tissue pH. In some implementations, the CT signal or image intensity provided by a gold or iodine nanoparticle allows for in vivo quantification of gadolinium, and thereby gadolinium nanoparticles, in each voxel, which can be used in conjunction with MRI measurements to determine tissue pH. In some embodiments, CT signal intensity correlates with computed tomography contrast agent concentration while an approximately constant MRI relaxivity is maintained. For example, an increase in CT signal intensity can correlate with an increase in computed tomography contrast agent concentration. As another example, a decrease in CT signal intensity can correlate with a decrease in computed tomography contrast agent concentration. Disclosed nanoparticles that can produce a concentration-dependent CT signal in conjunction with a relatively constant MRI signal can be used in conjunction with measurement of pH-dependent MRI signals to determine tissue pH.

The combination of MRI and CT imaging with a pH responsive polymer represents an improvement over known methods of measuring pHe, such as PET and OI, both of which are inaccurate and imprecise, magnetic resonance spectroscopy (MRS), the two-phase injection method, and chemical exchange saturation transfer (CEST) imaging.

The combination of MRI and CT imaging with a pH responsive polymer and a targeting agent also represents an improvement over known MRI-based methods of measuring pHe, which suffer from low changes in relaxivity and concomitantly limited sensitivity as well as poor specificity due to lack of molecular targeting. For example, dual-modality imaging using combined PET-MRI is hindered by a small molecule chelate structure, which limits the ability to add molecular targeting functionality, in contrast to the presently disclosed nanoparticles with targeting agents. Additionally, the PET-MRI method used radioisotopes, which is unfavorable both because it exposes patients to radioactive compounds and because it limits imaging time.

As described above, various diseases and disorders are characterized by an increase in pHe. Measurement of pHe using the disclosed nanoparticles can help determine the pHe of diseased cells or tissues. Determining pHe helps to detect and diagnose a disease state or determine if a therapeutic treatment has been effective. For example, gold/gadolinium or iodine/gadolinium MOF nanoparticles modified with pH responsive polymers and targeting agents can be used to measure the pHe of breast cancer tissue in vivo in a patient that has undergone a therapeutic treatment. An increase in pHe or a return to neutral pHe indicates that the treatment was effective, such as by decreasing the size of a tumor. A decrease in pHe indicates that the treatment was ineffective. By using the presently disclosed nanoparticles, pHe can be determined within a few days of administering a therapeutic treatment, which represents an improvement over known pHe-measuring techniques that do not provide feedback on the effectiveness or ineffectiveness of a therapy until after many weeks, such as 8 weeks, of treatment. The ability to provide feedback on a treatment and on a patient's health status, such as whether a tumor is shrinking, growing, or remaining unchanged during treatment, within a few days of treatment instead of many weeks helps to increase treatment options and improve outcomes for patients.

### EXAMPLES

The following examples are intended to be exemplary, and not limit, the present disclosure.

### Example 1

### Synthesis of Gold/Gadolinium Nanoparticles

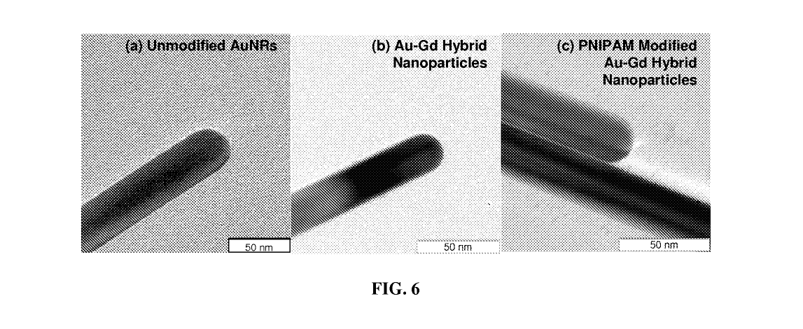

Gold nanoparticles were synthesized via procedures described herein. As shown in FIG. 6(a), one embodiment resulted in synthesized gold nanoparticles having an average length of 250 nm and width of 30 nm, providing an aspect ratio (length/width) of about 8 nm. Those of skill in the art will appreciate that the average dimensions of gold nanoparticles can be easily tuned with slight changes to the experimental procedures.

Gold nanoparticles were subsequently coated with a gadolinium -based nanoscale metal organic framework (NMOF). Coating of the gold nanoparticles was accomplished by taking advantage of a reverse microemulsion system, discussed in the literature and depicted in FIG. 1. In one embodiment reverse microemulsion provides gadolinium -based NMOFs. For gadolinium -based NMOFs, aqueous solutions of GdCl₃ (0.5M) and 1,4-benzenedicarboxylic acid (1,4-BDC) (0.075M) were first prepared separately. Next, the GdCl₃ and 1,4-BDC aqueous solutions were combined into a heptane/hexanol/cetyltrimethylammonium bromide (0.05M) microemulsion. This was followed by addition of the gold nanoparticles into the microemulsion reaction. The nanoparticle/gadolinium mixture was then stirred vigorously for 24 hours at room temperature.

After 24 h, the microemulsion mixture was centrifuged; the supernatant removed, and the nanoparticles subjected to three cycles of ethanol-wash/sonication/centrifugaton. The supernatant was collected and discarded. The gold/gadolinium nanoparticles were then subjected to one last wash in deionized water followed by centrifugation and then storage in fresh deionized water. The gold/gadolinium nanoparticles were characterized by TEM (transmission electron microscopy), UV-Vis (ultraviolet visual) spectroscopy, and ATR-FTIR (attenuated total reflection—fourier transform infrared).

TEM was used to visualize and measure the gadolinium-coated gold nanoparticles. TEM identified a uniform gadolinium-based coating of the gold nanoparticles with an average thickness of 4 nm (FIG. 6b).

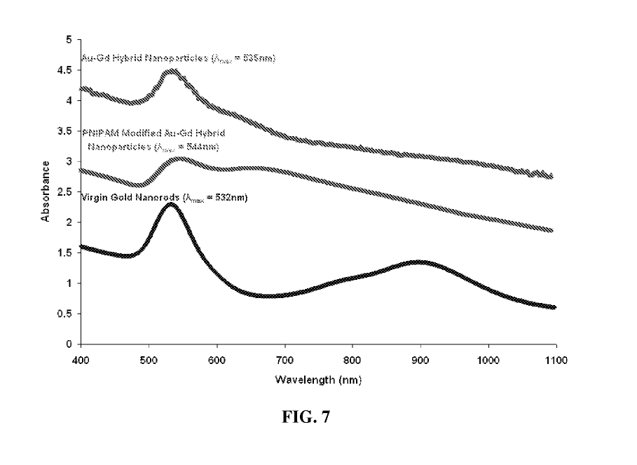

Ultraviolet-Violet spectroscopy was also used to analyze the gadolinium-coated gold nanoparticles. This technique was first used to analyze the virgin gold nanoparticles (FIG. 7), which gave a wavelength maximum for the transverse surface plasmon peak of 532 nm. Coating these particles with gadolinium-based MOF resulted in a red shift of the transverse surface plasmon peak maximum to 535 nm (FIG. 7). This 3 nm red shift correlated well to the thickness of the surface coating measured by TEM (FIG. 6b).

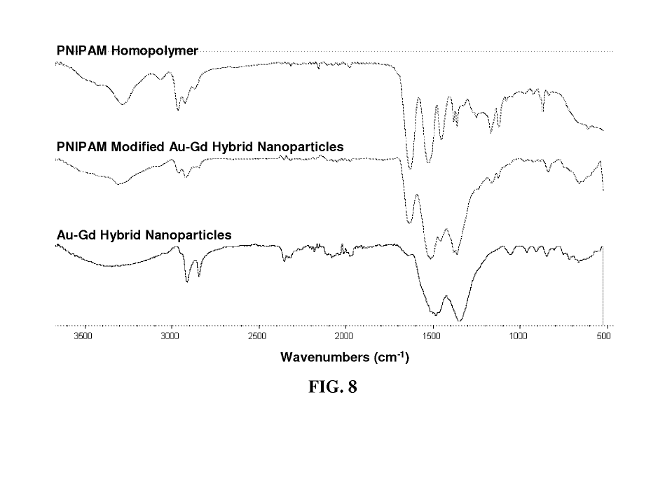

Finally, ATR-FTIR was also used to analyze the gadolinium coated nanoparticles. ATR-FTIR was used to probe the structure of the gadolinium-coated gold nanoparticles (FIG. 8). The gadolinium-coated gold nanoparticles produced a spectrum with a characteristic out-of-plane ═C—H aromatic stretch at 725 cm⁻¹, symmetric carboxylate stretch at 1450 cm⁻¹, an assymetric carboxylate stretch at 1520 cm⁻¹, along with 2855 cm⁻¹, 2925 cm⁻¹, and 3065 cm⁻¹. These peaks are attributed to the —C—H stretching vibrations of the 1,4-BDC bridging ligand ligand. There was also a 3460 cm⁻¹ peak which was attributed to the —OH stretch of the water ligand.

Thus these techniques confirmed the presence of a gadolinium coating on the gold nanoparticles that was measured to be approximately 3 to 4 nm thick.

### Example 2

### Synthesis of Iodine/Gadolinium Nanoparticles

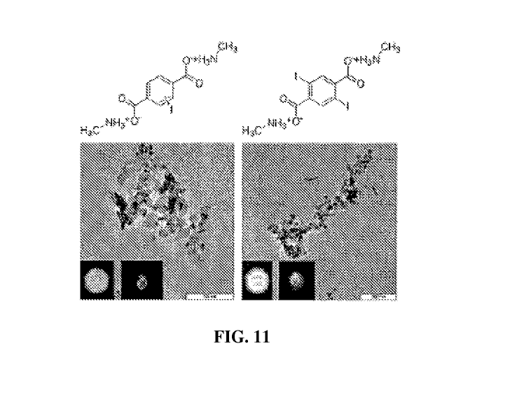

Iodine nanoparticles were synthesized according to procedures similar to Example 1. Nanoparticles were prepared using either a mono-iodo ligand (left panel, FIG. 11) or a di-iodo ligand (right panel, FIG. 11). The nanoparticles were evaluated by MRI and CT. Results are shown in FIG. 11. Results demonstrated that the nanoparticles behaved as a computed tomography for both MRI (lower left panel, left inset and lower right panel, left inset) and CT (lower left panel, right inset and lower right panel, right inset). These results also demonstrated an increased CT image intensity with increasing iodine concentration while maintaining an approximately constant MRI relaxivity.

### Example 3

### Polymer Surface Modification of Gold/Gadolinium Hybrid Nanoparticles

Scheme 1. Gold/gadolinium (Au/Gd) hybrid nanoparticles were modified with well-defined homopolymers and copolymers synthesized via reversible addition-fragmentation chain transfer (RAFT) polymerization. Without being limited to a specific mechanism or mode of action, one mechanism of attachment is shown in FIG. 1d. In the mechanism depicted in FIG. 1d, a thiolate terminated polymer is covalently attached to the nanoparticle surface through a coordination reaction between the polymer chain thiolate end-group moiety and vacant orbitals on the Gd³⁺ ions at the surface of the Gd nanoparticles.

In one embodiment, DATC was employed as the RAFT agent in the formation of homopolymers by RAFT polymerization. This DATC/RAFT reaction yields trithiocarbonate terminated chains. Au/Gd hybrid nanoparticles were modified with poly(N-isopropylacrylamide) (PNIPAM) homopolymer in N,N-dimethylformamide with the use of hexylamine as a reducing agent (Scheme 2). Modification of Au/Gd nanoparticles was achieved by an initial aminolysis, using hexylamine, of the trithiocarbonate end group of the RAFT homopolymer to a thiolate functionality under inert and basic conditions. Subsequently, the thiolate terminated homopolymer was covalently attached to the nanoparticle surface through a coordination reaction between the polymer and Gd³⁺.

After polymer deposition and prior to characterization or use, the nanoparticles were washed several times with a good solvent in order to remove any free polymer from the system.

The polymer modification of the Au/Gd nanoparticles with the PNIPAM homopolymer was analyzed by TEM (FIG. 6), UV-Vis spectroscopy (FIG. 7), and ATR-FTIR (FIG. 8). All three methods confirmed the presence of polymer modification on the nanoparticle.

TEM was again used to measure the thickness of the gold/gadolinium/polymer surface. This technique resulted in a measurement for the thickness of the gadolinium/polymer layer of approximately 8 nm. Subtracting out the apparent thickness of the gold/gadolinium layer (4 nm) suggested that the polymer layer was 4 nm thick. These values agreed well with results obtained by surface plasmon shift/UV-Vis spectroscopy.

FIG. 7 shows that the maximum surface plasmon peak of the gold/gadolinium/polymer nanoparticles occurred at 544 nm Comparing the UV/Vis spectra of gold nanoparticles, gold/gadolinium nanoparticles, and gold/gadolinium/polymer nanoparticles showed a 12 nm shift in surface plasmon peak between the gold nanoparticle and the gold/gadolinium/polymer nanoparticles. This represents a 9 nm red shift from gold/gadolinium nanoparticle to gold/gadolinium/polymer nanoparticle.

Finally, ATR-FTIR analysis identified significant N—H bonding and other changes in the spectrum of Au/Gd polymer nanoparticles compared to unmodified Au/Gd nanoparticles or homopolymer (FIG. 8). Several of the characteristic stretches of the free PNIPAM homopolymer, including a broad N—H stretch above 3300 cm⁻¹ and a small N—H bend at 1640 cm⁻¹ indicating the presence of the acrylamide functionality; an increase in intensity of the —CH2 stretching and C—H stretching vibrations between 2800-3000 cm⁻¹ due to backbone methylenes; a peak at 1720 cm⁻¹ assigned to the carbonyl stretch of the amide; and a stretch at 1380 cm⁻¹ attributed to the addition of —CH3 and isopropyl groups, display good transference to the polymer modified Au/Gd hybrid nanoparticles, when compared to the unmodified Au/Gd hybrid nanoparticles (FIG. 8).

Scheme 2. Surface Modification of Au/Gd Hybrid Nanoparticles by Incorporation of Optical Imaging Agents into Polymer Backbone.

Fluorescence imaging was used to analyze polymer modification of Au/Gd nanoparticles. Incorporation of a poly(fluorescein O-methacrylate) monomer into the backbone of the copolymer would provide a means for measuring polymer incorporation through the use of in vitro fluorescence imaging. To do this, Au/Gd hybrid nanoparticles were first modified with a fluorescent RAFT copolymer, PNIPAM-co-poly(N-acryloxysuccinmide)(PNAOS)-co-poly(fluorescein O-methacrylate) (PFMA).

A fluorescence scanner was used in order to confirm incorporation and provide images of fluorescent gold/Gd nanoparticles. FIG. 5 is a fluorescence image of gold/gadolinium nanoparticles coated with a fluorescent copolymer.

### Example 4

### Effect of pH on Relaxivity of Nanoparticles

Gadolinium MOF nanoparticles were modified with pH responsive or non-pH responsive polymers via RAFT polymerization similarly to the procedure described in Example 3. The modified Gd MOF nanoparticles were evaluated by MRI. Results demonstrated that for the Gd MOF nanoparticles surface-modified with a non-pH responsive polymer, changing the pH from 6 to 3 resulted in a 0.8% change in the longitudinal relaxation time (T1). For the Gd MOF nanoparticles surface-modified with a pH responsive polymer, changing the pH from 6 to 3 resulted in a 58% change in T1. Thus, under acidic conditions, a nanoparticle modified with a pH responsive polymer demonstrated a greater than 70-fold increase in the effect on T1 compared to a non-pH responsive polymer.

### Example 5

### Biocompatibilty of Gold/Gadolinium Nanoparticles.

The ability of modified and unmodified Au/Gd nanoparticles to inhibit cell growth was compared in vitro.

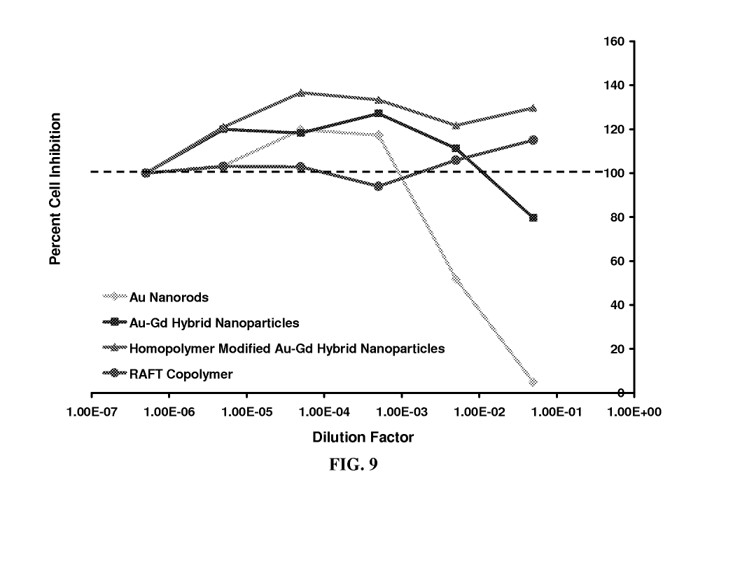

To test the biocompatibility of the unmodified and polymer modified gold/Gd nanoparticles, growth inhibition studies were performed using a canine endothelial hemangiosarcoma (FITZ-HSA) tumor cells. Samples were incubated with FITZ-HSA at 37° C. in standard culture medium containing 10% PBS for 72 h in a 5% CO₂ atmosphere. Each of the components for the nanodevice synthesis, gold nanoparticles, RAFT copolymer, along with the unmodified and polymer-modified gold/Gd nanoparticles were tested. As can be seen in FIG. 9, the unmodified gold nanoparticles resulted in significant cell growth inhibition at high concentrations. This is most likely due to residual CTAB absorbed on the surface of the gold nanoparticles. However, note that modification of the gold nanoparticles to form the gold/Gd nanoparticles and further modification of the gold/Gd nanoparticles did not decrease cell viability. The increased cell viability is attributed to coating of the gold/Gd nanoparticles with copolymers consisting of the biocompatible polymer PNIPAM. This infers that the presence of the RAFT copolymer on the surface of the gold/Gd nnaoparticles increases the biocompatible nature of the nanodevice.

In vitro MRI of Gadolinium Nanoparticles and Polymer Modified Gadolinium Nanoparticles. In order to provide information about the clinical imaging viability of the polymer modified gold/Gd nanoparticles as a positive contrast agent, in vitro MRI was employed to determine relaxation properties of the unmodified and polymer modified gold/Gd nanoparticles. Table 1 compares the MRI longitudinal and transverse relaxivities, r1 and r2, respectively, of PNIPAM-modified gold/Gd nanoparticles and gold/Gd unmodified nanoparticles to the clinically employed contrasts agents, gadopentetate dimeglumine (Magnevist®) and gadobenate dimeglumine (Multihance®). The calculated relaxivities demonstrate that both the unmodified and polymer modified Gd nanoparticles result in a large shortening of the T1 relaxation time and, thus, behave as positive contrast agents. Additionally, the ratio of the transverse and longitudinal relaxivities of the unmodified and polymer modified gold/Gd nanoparticles are less than that of the clinically employed contrast agents, Magnevist® and Multihance®, suggesting that unmodified and polymer-modified Gd nanoparticles should produce potentially feasible clinically useful T1 shortening effects, in comparison to currently employed contrast agents. By taking advantage of these properties, novel theragnostic polymer-modified gold/Gd nanoparticles could be produced and exploited as contrast agents for conventional T1 MR imaging.

TABLE 1
MRI relaxivity values for clinical MRI contrast agents,
Multihance ® and Magnevist ®, along with the unmodified
and polymer modified gold/Gd nanoparticles.

| Contrast Agent | r₁ (mM/L) | r₂ (mM/L) | r₂/r₁ |
|---|---|---|---|
| Magnevist ® | 6.95 | 17.41 | 2.51 |
| Multihance ® | 17.70 | 35.57 | 2.01 |
| Unmodified Au—Gd Hybrid NPs | 6.08 | 8.22 | 1.35 |
| PNIPAM Homopolymer Modified | 11.28 | 18.83 | 1.67 |
| Au—Gd Hybrid NPs | | | |
| PNIPAM Copolymer Modified | 13.55 | 21.85 | 1.61 |
| Au—Gd Hybrid NPs | | | |

Applicants further note that the compounds and methods disclosed herein include those compounds cited in U.S. 2007/0123670 to McCormick et al., which is incorporated herein by reference in its entirety.

All references cited herein are incorporated herein by reference in their entirety.

What is claimed is:

1. A nanoparticle conjugate comprising:
a gadolinium metal organic framework disposed on a nanoparticle comprising a computed tomography contrast agent; and
at least one pH responsive polymer bound to the nanoparticle.

2. The nanoparticle conjugate of claim 1, wherein the computed tomography contrast agent comprises iodine.

3. The nanoparticle conjugate of claim 1, wherein the computed tomography contrast agent comprises gold.

4. The nanoparticle conjugate of claim 1, wherein the pH responsive polymer is configured to allow water to access or limit water from accessing the gadolinium metal organic framework.

5. The nanoparticle conjugate of claim 4, wherein allowing water to access the gadolinium metal organic framework increases relaxivity of the nanoparticle in magnetic resonance imaging.

6. The nanoparticle conjugate of claim 1, wherein longitudinal relaxation time of the nanoparticle conjugate changes as pH changes.

7. The nanoparticle conjugate of claim 6, wherein longitudinal relaxation time decreases when pH decreases.

8. The nanoparticle conjugate of claim 1, comprising a targeting agent configured to target the nanoparticle to a cell.

9. The nanoparticle conjugate of claim 1, wherein the pH responsive polymer is bound to the nanoparticle via a polymer, polymer precursor, or initiator grafted onto the gadolinium metal organic framework.

10. A method of making a nanoparticle conjugate comprising:
disposing a gadolinium metal organic framework on a nanoparticle comprising a computed tomography contrast agent; and
operably associating at least one pH responsive polymer with the nanoparticle.

11. The method of claim 10, wherein the computed tomography contrast agent comprises iodine or gold.

12. The method of claim 10, wherein the pH responsive polymer is bound to the gadolinium metal organic framework nanoparticle by reversible addition-fragmentation chain transfer polymerization.

13. The method of claim 10, further comprising operably associating at least one targeting agent configured to target the nanoparticle to a cell.

14. A method of determining extracellular pH in vivo comprising:
administering a gadolinium metal organic framework nanoparticle comprising a computed tomography contrast agent and polymers bound to the computed tomography contrast agent to a patient, wherein at least a portion of the polymers target the nanoparticles to cells of interest and at least a portion of the polymers are pH responsive;
measuring longitudinal relaxation time of the nanoparticles by magnetic resonance imaging, wherein a change in relaxation time indicates extracellular pH; and
quantifying the nanoparticles by imaging the nanoparticles with computed tomography, wherein signal intensity correlates with computed tomography contrast agent concentration.

15. The method of claim 14, wherein the computed tomography contrast agent is iodine.

16. The method of claim 14, wherein the computed tomography contrast agent is gold.

17. The method of claim 14, wherein an increase in computed tomography signal intensity correlates with an increase in computed tomography contrast agent concentration.

18. A method of characterizing cells in a patient comprising:
administering a gadolinium metal organic framework nanoparticle comprising a computed tomography contrast agent and comprising operably associated polymers to a patient, wherein at least a portion of the polymers target the nanoparticles to cells of interest and at least a portion of the polymers are pH responsive; and
measuring longitudinal relaxation time of the nanoparticles by magnetic resonance imaging, wherein a change in relaxation time indicates extracellular pH,
wherein an acidic extracellular pH indicates diseased cells.

19. The method of claim 18, wherein the computed tomography contrast agent is iodine.

20. The method of claim 18, wherein the computed tomography contrast agent is gold.

21. The method of claim 18, further comprising quantifying the nanoparticles by imaging the nanoparticles with computed tomography, wherein signal intensity correlates with computed tomography contrast agent concentration.

22. The method of claim 21, wherein an increase in signal intensity correlates with an increase in computed tomography contrast agent concentration.
